(19) United States
(12) Patent Application Publication
(10) Pub. No.: US 2011/0293553 A1
(43) Pub. Date: Dec. 1, 2011

(54) IDENTIFICATION OF INSECT ATTRACTANT, ARRESTING, AND/OR AGGREGATION COMPOUNDS AND METHODS THEREOF

(76) Inventors: Tai-Teh Wu, Chapel Hill, NC (US); Kenneth Haynes, Lexington, KY (US); Joe Harold Hope, III, Mebane, NC (US)

(21) Appl. No.: 12/790,168

(22) Filed: May 28, 2010

(51) Int. Cl. A01N 35/10; C07C 251/08; C07C 251/76; C07C 251/48; C07C 251/44; C07C 49/04; C07D 257/08; A01N 31/02; A01N 43/713; A01N 43/88; A01P 7/04; A01P 19/00; G01N 30/02; G01R 33/44; H01J 49/26; C07C 251/36

(52) U.S. Cl. 424/84; 564/268; 564/278; 564/250; 564/267; 564/253; 568/382; 544/179; 436/161; 324/309; 250/282

(57) ABSTRACT

Compounds, compositions, kits, devices, and methods of attracting, detecting, eradicating, controlling, or killing an insect, such as a bed bug, by utilizing insect attractant, arresting, and/or aggregation compounds and compositions is provided. Insect attractant, arresting, and/or aggregation compounds identified from insect fecal extract by an analytical technique, such as gas chromatography, nuclear magnetic resonance (NMR), Carbon-13 NMR, mass spectroscopy, LC-MS, GC-MS, high performance liquid chromatography (HPLC), or combinations thereof are provided. A bed bug attractant, arresting, and/or aggregation compound identified from bed bug feces and exhibiting a Carbon-13 NMR peak at about δ 159.453 ppm is also provided.

### FIELD

This disclosure relates to the identification of an insect attractant, arresting, and/or aggregation compound and the use of a compound in methods, compositions, detection devices, and traps. In an aspect, the insect is a bed bug.

### BACKGROUND

Bed bugs are small insects that feed solely on the blood of animals. The common bed bug, Cimex lectularius, is the species of bed bug that has most adapted to living with human beings. Adult bed bugs are about ¼-inch or about 6 millimeters long, 5 to 6 millimeters wide, and reddish-brown with oval, flattened bodies. The immature nymphs are similar in appearance to the adults but smaller and lighter in color.

Bed bugs do not fly but they can move very quickly over surfaces. Female bed bugs lay their eggs in secluded areas and can deposit up to five eggs per day, and as many as 500 during a lifetime. Bed bugs can survive for extended periods without feeding. For example, nymphs can survive months without feeding and the adults can survive more than a year. As a result, infestations are not likely to be eliminated by the absence of a host in the area of concern.

Bed bugs obtain their sustenance by drawing blood through an elongated beak. They may feed on a human being for 3 to 10 minutes, although the human being is not likely to feel the bite. After the bite, the human victim often experiences an itchy welt or swelling in the area of the bite. Because some victims do not have any reaction, or only a minimal reaction to a bed bug bite, infestations may go long periods without being detected. New bed bug infestations originate from a bed bug being carried into a new area, such as by clinging to possessions which are easily transported, for example, clothing, sheets, and other items. Bed bug bugs are also commonly introduced to new areas on used furniture, mattresses, or by way of suitcases. Additionally, the ability of bed bugs to disperse by walking can spread an infestation between nearby units in a multi-unit dwelling (such apartments, nursing homes, and motel/hotel rooms). As a consequence, living areas where the turnover of occupants is high, such as hotels or apartments, are especially vulnerable to bed bug infestations.

For the foregoing reasons, bed bugs are often difficult to detect and eradicate. Pest management professionals (PMPs) and pesticides are typically required, necessitating removal of non-essential objects from a room, removal of bed bugs and eggs through vacuuming and/or steaming and then application of pesticides to likely hiding areas. Because treatment for bed bug infestation is intrusive, disruptive to normal business operations and expensive, early detection and treatment of bed bugs and continuous monitoring for the presence of bed bugs is highly desirable. If early detection is made, appropriate steps can be taken before infestation becomes established.

What is needed, therefore, is a detection device for monitoring bed bug infestation which is attractive to bed bugs, easy to handle, discrete, and deployable across a wide range of areas, for example, in hotels and other lodging locations that experience frequent turnover of occupancy. Also needed are compounds and compositions capable of attracting insects and treating insect infestation. Such compounds and compositions may be used together with devices to detect insect infestation, with traps to capture insects for detection or mitigate of infestation, or with insecticides to treat infestations.

### SUMMARY

The disclosure provides a method for identifying a compound from insect feces or feces extract. In another aspect, a compound isolated or identified from insect feces or feces extract is capable of attracting, arresting, aggregating, or controlling an insect. In an aspect, the insect is a bed bug.

The disclosure also provides for a method of identifying an insect attractant, arresting, and/or aggregation compound from insect feces by an analytical technique, such as gas chromatography, nuclear magnetic resonance (NMR), mass spectroscopy, liquid chromatography-mass spectrometry (LC-MS), gas chromatography-mass spectrometry (GC-MS), high performance liquid chromatography (HPLC), and/or combinations thereof.

In an aspect, the disclosure provides for a compound identified or isolated from insect feces. In another aspect, a compound identified or isolated from insect feces exhibits a peak from about δ 155 ppm to about δ 165 when analyzed by Carbon-13 NMR. In another aspect, a compound identified or isolated from insect feces exhibits a peak at about δ 160 ppm when analyzed by Carbon-13 NMR. In another aspect, a compound identified or isolated from insect feces exhibits a peak at about δ 159.453 ppm when analyzed by Carbon-13 NMR. In yet another aspect, a compound identified or isolated from insect feces exhibits a peak at about δ 159.453 ppm when analyzed by Carbon-13 NMR and comprises an oxime, hydrazone, imino, amidine, or imine functional group. The disclosure also provides for a compound identified or isolated from bed bug feces that exhibits a peak at about δ 159.453 ppm when analyzed by Carbon-13 NMR and comprises from about five to ten carbon atoms and an oxime group functional group. In another aspect, a compound exhibits the Carbon-13 NMR structure as set forth in FIGS. 3, 4, 5, 6, 7, and/or 8. In yet another aspect, a compound identified by the above parameters is capable of attracting, arresting, aggregating, controlling, and/or influencing the behavior of an insect, such as a bed bug.

In another aspect, the disclosure provides for methods of attracting, detecting, eradicating, controlling, and/or killing an insect by utilizing a compound identified from insect feces as described herein. The disclosure also provides for compositions, kits, and devices comprising compounds identified from insect feces.

The disclosure also provides for bait compositions comprising a compound described herein. In an aspect, the disclosure provides for a method of treating insect infestation and/or determining the presence of an insect, by providing a bait material comprising a compound identified from insect feces. The bait composition may be used in any of the methods described herein and may optionally include an insecticide.

In another aspect, the disclosure provides for a detection device or trap containing a compound described herein. In another aspect, the disclosure provides for a detection device or trap containing a compound identified or isolated from insect feces. In an aspect, a bait composition can be used with the detection device or trap. In an aspect, the disclosure provides for a detection device for monitoring the presence of bed bugs and/or infestation, comprising a first plate and a second plate, wherein the first plate and the second plate are separated by an internal spacing sized to permit entry into the detection device by one or more bed bugs; and a compound described herein.

In an aspect, the disclosure provides for a support material comprising a compound described herein. In one aspect, the support material is coated or impregnated with a compound or composition described herein. The support material may be, for example, an absorbent material, wood, cardboard, corrugated cardboard, cotton, wallpaper, paper, plastic, a plastic tube, or metal.

The disclosure also provides for a method of luring, arresting, and/or aggregating an insect into an area treated with an insect attractant, arresting, and/or aggregation compound identified or isolated from insect feces. In one aspect, the treated area is a support material capable of being coated or impregnated with a compound identified or isolated from insect feces.

In an aspect, an insect attractant, arresting, and/or aggregation compound or composition identified or isolated from insect feces described herein may be combined with an insecticide or pheromone. In another aspect, the insecticide is selected from the group consisting of carbamates, pyrethroids, phenylpyrazoles, such as fipronil or ethiprole, chloronicotinyles, imidacloprid, acetamiprid, thiacloprid, thiamethoxam, nitenpyram, clothianidin, oxadiazines, anthranilic diamides, butenolides, sulfoximines, indoxacarb, and rynaxypyr. In yet another aspect, compositions, methods, and/or devices described herein do not include an insecticide.

In an aspect, the insect in the above methods is a bed bug.

Additional objects, features and advantages of the invention will be set forth in the description which follows, and in part, will be obvious from the description, or may be learned by practice of the invention. The objects, features and advantages of the invention may be realized and obtained by means of the instrumentalities and combination particularly pointed out in the appended claims.

### BRIEF DESCRIPTION OF THE DRAWINGS

FIG. 1 provides a fractionation scheme of bed bug feces extract in conjunction with flash chromatography.

FIG. 2 provides a Gas Chromatography profile of extracted bed bug feces.

FIG. 3 provides a full C-13 NMR spectrum of a bed bug feces extract.

FIG. 4 provides a C-13 NMR scan from 0 to 80 ppm of a bed bug feces extract.

FIG. 5 provides a C-13 NMR scan from 170 to 118 ppm of a bed bug feces extract.

FIG. 6 provides a C-13 NMR scan from 72 to 31 ppm of a bed bug feces extract.

FIG. 7 provides a C-13 NMR scan from 30 to 10 ppm of a bed bug feces extract.

FIG. 8 provides the peak frequency and intensity of the C-13 NMR scan of a bed bug feces extract.

FIG. 9 provides an overview of a two-choice bioassay experiment.

FIG. 10 provides a perspective view of a detection device.

FIG. 11 provides for a liquid chromatography-mass spectrometry (LC-MS) spectrum of a bed bug feces extract.

FIG. 12 provides for an LC-MS spectrum on a solvent blank.

FIG. 13 provides for a LC-MS spectrum at a retention time at 0.489 min of a bed bug feces extract.

FIG. 14 provides for a LC-MS spectrum at a retention time at 0.558 min of a bed bug feces extract.

FIG. 15 provides for a LC-MS spectrum at a retention time at 0.593 min of a bed bug feces extract.

FIG. 16 provides for a LC-MS spectrum at a retention time at 4.794 min of a bed bug feces extract.

FIG. 17 provides for a LC-MS spectrum at a retention time at 4.722 min of a bed bug feces extract.

FIG. 18 provides for a LC-MS spectrum at a retention time at 4.253 min of a bed bug feces extract.

FIG. 19 provides for a LC-MS spectrum at a retention time at 5.034 min of a bed bug feces extract.

FIG. 20 provides for a LC-MS spectrum at a retention time at 5.107 min of a bed bug feces extract.

FIG. 21 provides for a LC-MS spectrum at a retention time at 5.197 min of a bed bug feces extract.

FIG. 22 provides for a LC-MS spectrum at a retention time at 5.374 min of a bed bug feces extract.

FIG. 23 provides for a LC-MS spectrum at a retention time at 5.863 min of a bed bug feces extract.

### DETAILED DESCRIPTION

The disclosure provides a method for identifying a compound from insect feces, wherein the compound is capable of attracting, arresting, and/or aggregating an insect. In an aspect, the insect feces may be dissolved in a solvent thereby creating a fecal extract prior to identifying the compound. When dissolved in a solvent, a fecal extract can be obtained.

In another aspect, the bed bug is from the species Cimex lectularius. While the instant disclosure focuses on utilizing the feces or fecal extract of bed bugs for identification of suitable compounds to utilize attractants, arresting, and/or aggregation compounds as well as compounds that influence the behavior of bed bugs, the instant disclosure could well be translated to be utilized with other related, blood-feeding arthropods or insects because species that share a similar food source and/or similar benefits of aggregations and are related may also share common attractants, arrestants, aggregation compounds or other pheromones.

The disclosure also provides for a method of identifying a compound from an insect by:
(1) analyzing and/or collecting feces from an insect;
(2) identifying a compound from the feces.

The disclosure also provides for a method of identifying an insect attractant, arrestant, or aggregation compound by:
(1) placing an insect on a support material;
(2) analyzing and/or collecting feces from an insect;
(3) identifying a compound from the feces.

In another aspect, the disclosure provides for a method of identifying a compound from an insect by:
(1) placing an insect on a support material;
(2) collecting feces from an insect;
(3) dissolving the collected feces in a solvent thereby obtaining fecal extract; and
(4) identifying a compound from the fecal extract.

The disclosure also provides for a method of identifying a compound, comprising analyzing insect feces or a fecal extract by a method selected from the group consisting of gas chromatography, NMR, Carbon-13 NMR, HPLC, mass spectroscopy, or LC-MS, GC-MS, or combinations thereof. In another aspect, the disclosure provides for a method of identifying an a compound, comprising analyzing insect feces or a fecal extract by gas chromatography together in combination with NMR, HPLC in combination with NMR, mass spectroscopy in combination with NMR, GC-MS in combination with NMR, or LC-MS in combination with NMR, or combinations thereof. In yet another aspect, a compound identified by the method is capable of attracting, arresting, aggregating, controlling, and/or influencing the behavior of an insect, such as a bed bug.

The disclosure also provides for a method of identifying an insect attractant, arrestant, and/or aggregation compound, comprising:
(1) collecting feces from an insect; and
(2) analyzing the feces by a method selected from the group consisting of gas chromatography, NMR, HPLC, LC-MS, GC-MS, and mass spectroscopy to thereby identify a compound capable of attracting, arresting, or aggregating an insect.

In another aspect, the collected feces are dissolved in a solvent thereby obtaining fecal extract prior to analysis by gas chromatography, NMR, HPLC, LC-MS, GC-MS, and/or mass spectroscopy.

In another aspect, the fecal extract is tested for the ability to attract, arrest, aggregate, or control an insect before or after analysis by gas chromatography, NMR, Carbon-13 NMR, HPLC, LC-MS, GC-MS, or mass spectroscopy.

The disclosure also provides for a method of identifying an insect attractant, comprising analyzing insect feces or insect fecal extract by Carbon-13 NMR, thereby identifying a compound exhibiting a Carbon-13 NMR peak at about δ 159.453 ppm, wherein the identified compound exhibiting a Carbon-13 NMR peak at about δ 159.453 ppm is capable of attracting an insect.

In an aspect, the disclosure also provides for a method of identifying a compound, comprising:
(1) Analyzing and/or collecting feces from an insect;
(2) dissolving the feces in a solvent thereby obtaining fecal extract; and
(3) analyzing the fecal extract by Carbon-13 NMR to thereby identify a compound exhibiting a Carbon-13 NMR peak at about δ 159.453 ppm.

In another aspect, the identified compound exhibiting a Carbon-13 NMR peak at about δ 159.453 ppm is capable of attracting, arresting, aggregating, controlling, or influencing the behavior of an insect.

The disclosure also provides for a method of controlling or detecting an insect, comprising providing a trap or detection device with a compound identified by the following method:
(1) analyzing and/or collecting feces from an insect;
(2) dissolving the feces in a solvent thereby obtaining fecal extract; and
(3) identifying a compound from the fecal extract;

wherein the identified compound is capable of controlling or detecting an insect.

The disclosure also provides for a method of attracting, arresting, or aggregating an insect, comprising providing a support material, bait composition, or detection device with a compound identified by the following method:
(1) collecting feces from an insect; and
(2) identifying a compound from the fecal extract;

wherein the identified compound is capable of attracting, arresting, or aggregating an insect.

The disclosure also provides for a method for isolating or identifying a bed bug attractant, arrestant, or aggregation compound, comprising:
(1) placing an insect on a support material;
(2) collecting feces from the insect;
(3) dissolving the collected feces in a solvent thereby obtaining fecal extract; and
(4) separating a compound from the fecal extract;

wherein the isolated compound is capable of attracting, arresting, or aggregating a bed bug.

In an aspect, the disclosure also provides for a method for isolating a bed bug attractant, arrestant, or aggregation compound, comprising:
(1) placing a bed bug on a support material;
(2) collecting feces from the bed bug; and
(3) separating a compound from the feces;

wherein the isolated compound is capable of attracting, arresting, or aggregating a bed bug.

In an aspect, the disclosure also provides for a method of controlling a bed bug, comprising providing a trap or detection device with a compound isolated by the following method:
(1) collecting feces from a bed bug;
(2) dissolving the feces in a solvent thereby obtaining fecal extract; and
(3) isolating a compound from the fecal extract;

wherein the isolated compound is capable of controlling a bed bug.

The disclosure further provides for a support material, bait material, detection device, or trap with a compound isolated or identified from insect feces, wherein the isolated or identified compound is capable of attracting, arresting, or aggregating an insect.

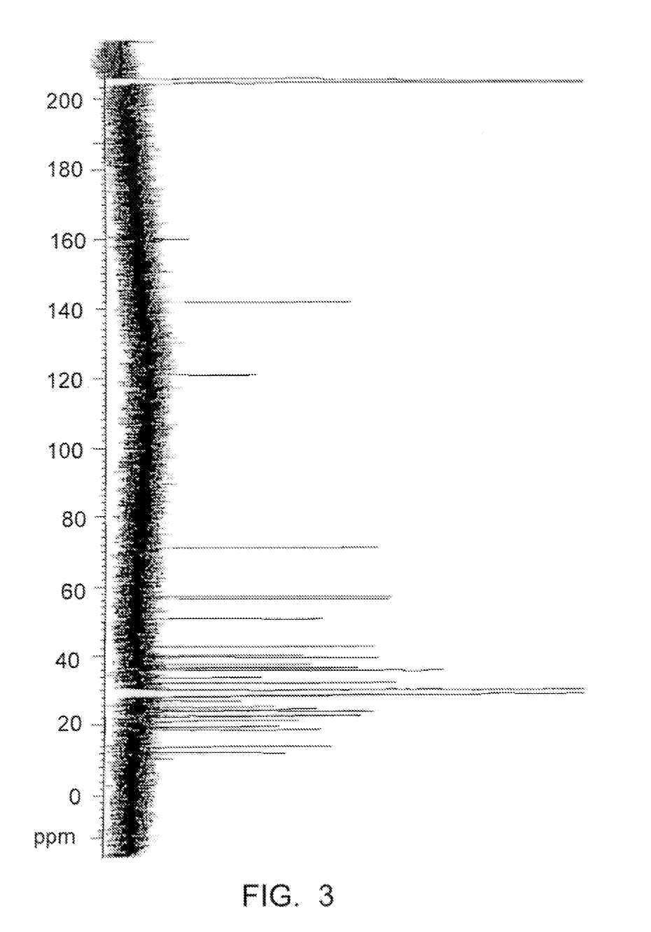

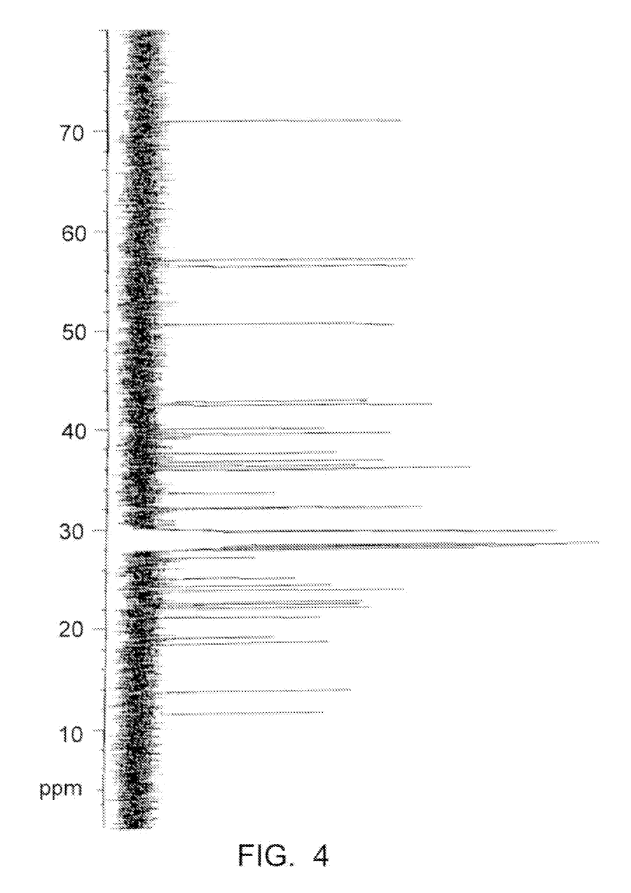

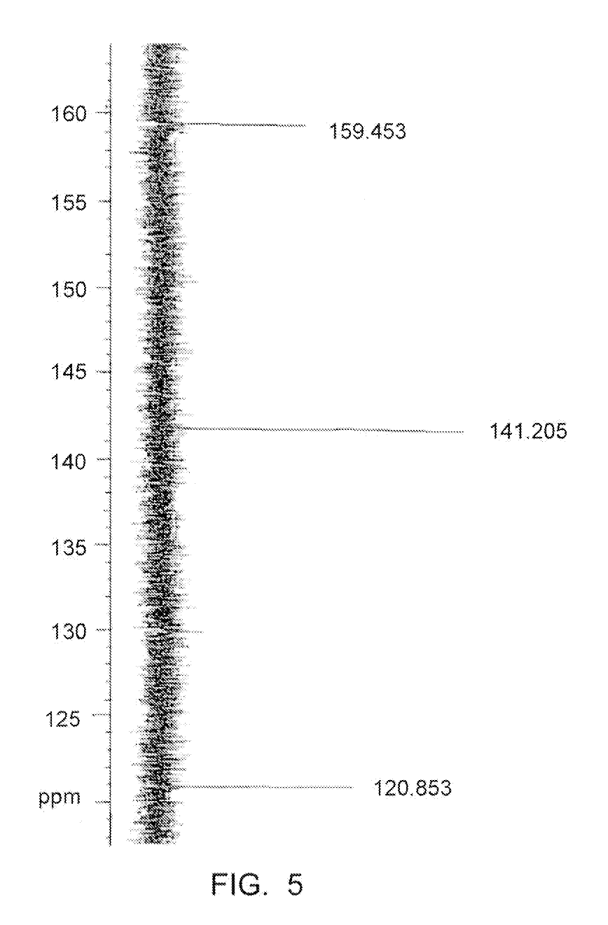

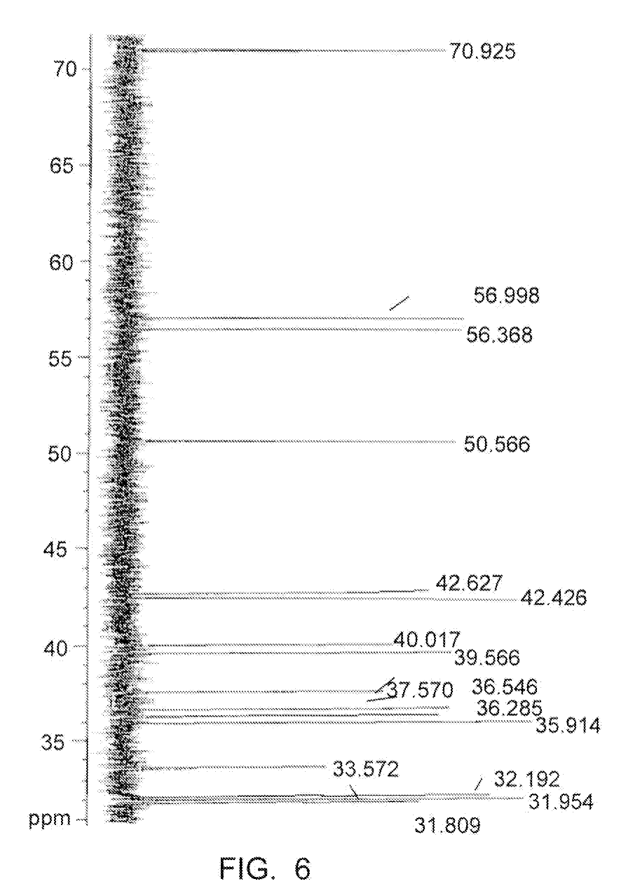

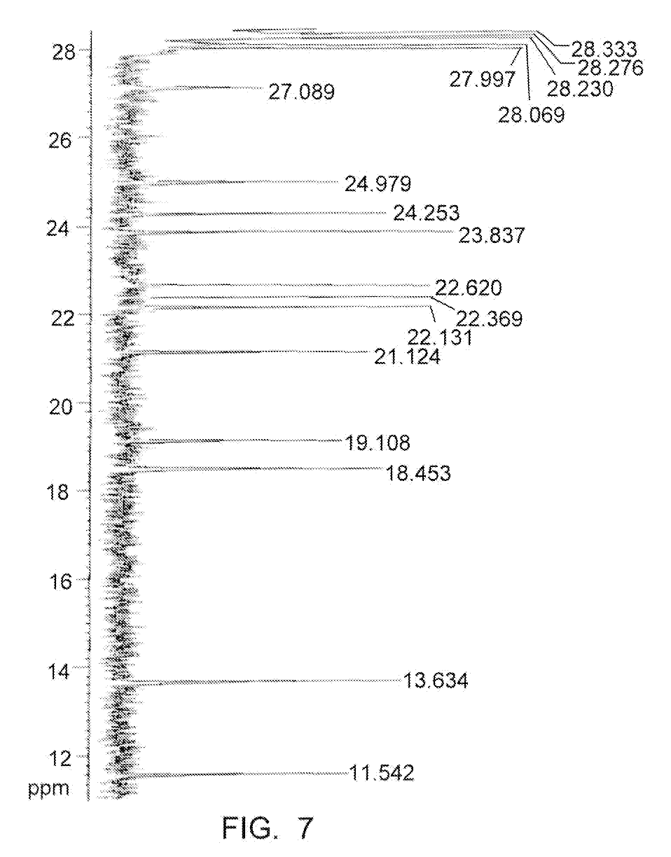

The disclosure further provides for compositions, bait compositions, devices, support materials, and methods comprising a compound exhibiting a Carbon 13 NMR spectrum as set forth in FIGS. 3, 4, 5, 6, 7 and/or 8. In another aspect, a compound described herein exhibits a peak at about δ 159.5 ppm, about δ 160 ppm, and/or from about δ 155 ppm-δ 165 ppm when analyzed by Carbon-13 NMR. In yet another aspect, a compound identified by the above parameters is capable of attracting, arresting, aggregating, controlling, and/or influencing the behavior of an insect, such as a bed bug.

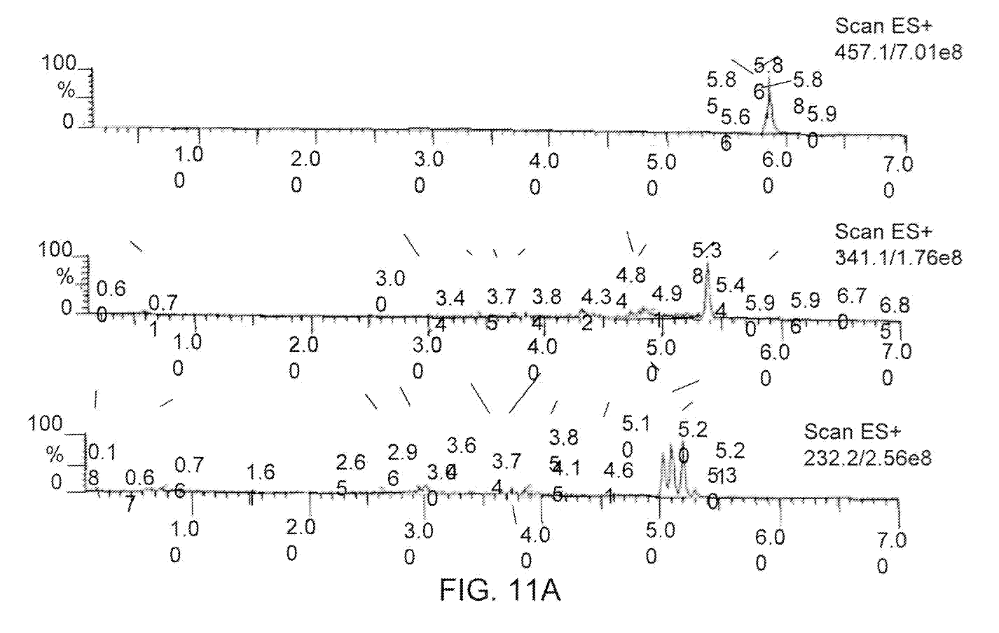

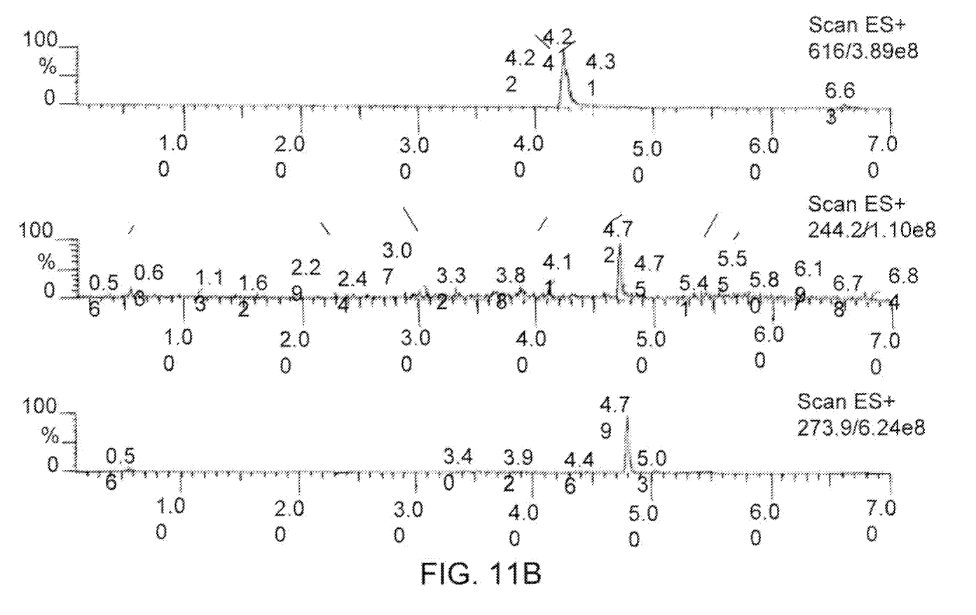

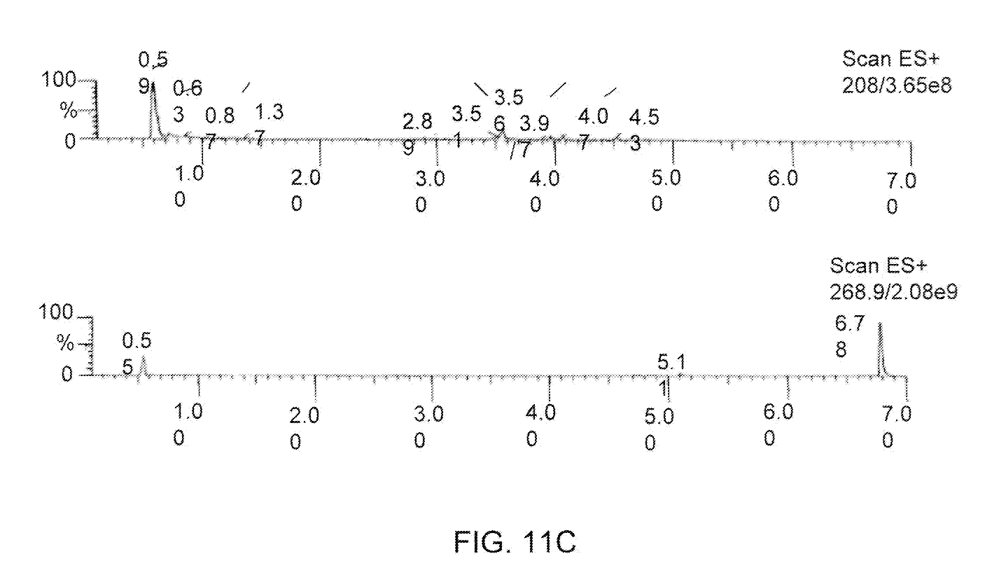

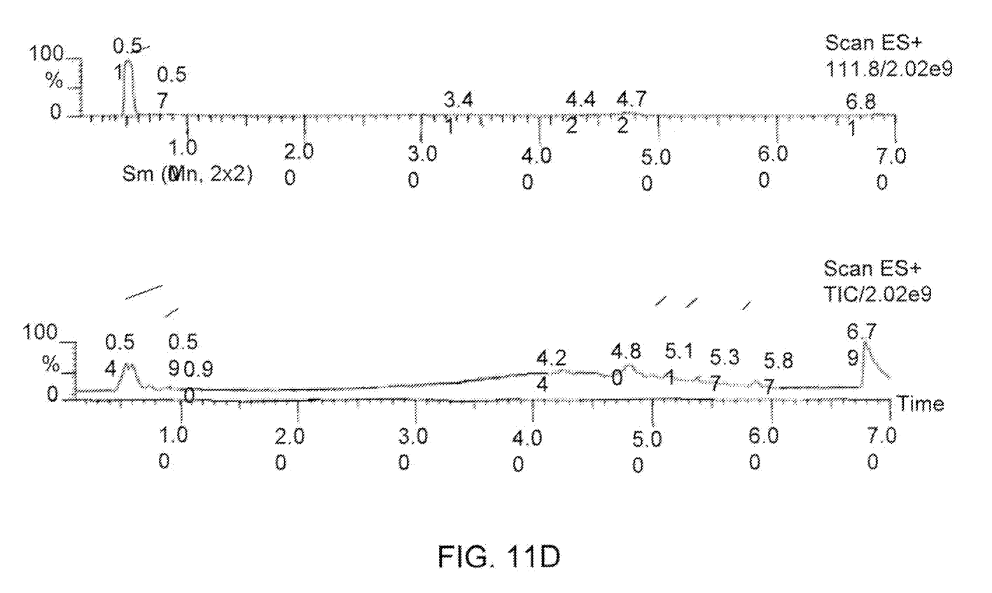

In another aspect, the disclosure provides for compositions, bait compositions, devices, support materials, and methods comprising a compound exhibiting a LC-MS spectrum as set forth in FIGS. 11-23.

The disclosure further provides for a support material, bait material, detection device, or trap with a compound isolated by the following method:
(1) analyzing and/or collecting feces from a bed bug;
(2) isolating a compound from the fecal extract.

As applied to the above methods, compositions, or devices, a insect attractant, arresting, and/or aggregation compound may be indentified by one or more analytical techniques, for example, gas chromatography, nuclear magnetic resonance (NMR), LC-MS, GC-MS, mass spectroscopy, or high performance liquid chromatography (HPLC), or any other technique capable of identifying or isolating a compound from feces or fecal extract.

As applied to the above methods, compositions, or devices, a insect attractant, arresting, and/or aggregation compound may be identified by Carbon-13 NMR. In yet another aspect, the solvent is removed from feces or fecal extract prior to analysis with Carbon-13 NMR. Generally, the solvent can be evaporated from the fecal extract under a variety of conditions based on sample volume, vacuum or hood conditions, the type of solvent that is evaporated, and temperature. One of ordinary skill in the art would readily recognize that sample volume also influences the amount of time necessary to evaporate a given solvent. According to the disclosure, the solvent can be evaporated from the fecal extract for two days at room temperature prior to analysis with Carbon-13 NMR. In another aspect, the solvent can be evaporated from the fecal extract for a time period of about 1 hour, about 2 hours, about 5 hours, about 12 hours, about 1-2 days, about 1 to about 5 days, or about 7 days or more. Any of the above evaporation times/techniques may also be used in conjunction with, for instance, the methodology of Example 4.

In another aspect, an insect attractant, arresting, and/or aggregation compound identified or isolated from insect feces exhibits a peak at about δ 158 ppm to about δ 162 ppm, about δ 155 ppm to about 67 165 ppm, about δ 155 ppm, about δ 156 ppm, about δ 157 ppm, about δ 158 ppm, about δ 159 ppm, about δ 159.5 ppm, about δ 160 ppm, about δ 161 ppm, about δ 162 ppm, about δ 163 ppm, about δ 164 ppm, or about δ 165 ppm when analyzed by NMR, for example, Carbon-13 NMR. In another aspect, insect attractant, arresting, and/or aggregation compound identified or isolated from insect feces exhibits a peak at about δ 159.453 ppm when analyzed by Carbon-13 NMR. Such a compound is capable of attracting, aggregating, arresting, or influencing the behavior of insects, such as bed bugs, and can be used in bait compositions, detection devices, traps, or with any method described herein.

In another aspect, a compound identified or isolated from insect feces or fecal extract and exhibiting at least one of the above peaks when evaluated by NMR, for example Carbon-13 NMR, comprises an oxime, a hydrazone, imino, amidine, or an imine functional group. In yet another aspect, a compound identified or isolated from insect feces or fecal extract and evaluated by NMR, for example Carbon-13 NMR, comprising from four, five, six, seven, eight, nine, ten, or eleven carbons or about five to ten carbon atoms and a functional group selected from oxime, a functional group selected from an oxime, a hydrazone, imino, amidine, or an imine. The disclosure also provides for a compound identified or isolated from insect feces or fecal extract and evaluated by NMR, for example Carbon-13 NMR, comprising from four, five, six, seven, eight, nine, ten, or eleven carbons or about five to ten carbon atoms and an oxime functional group.

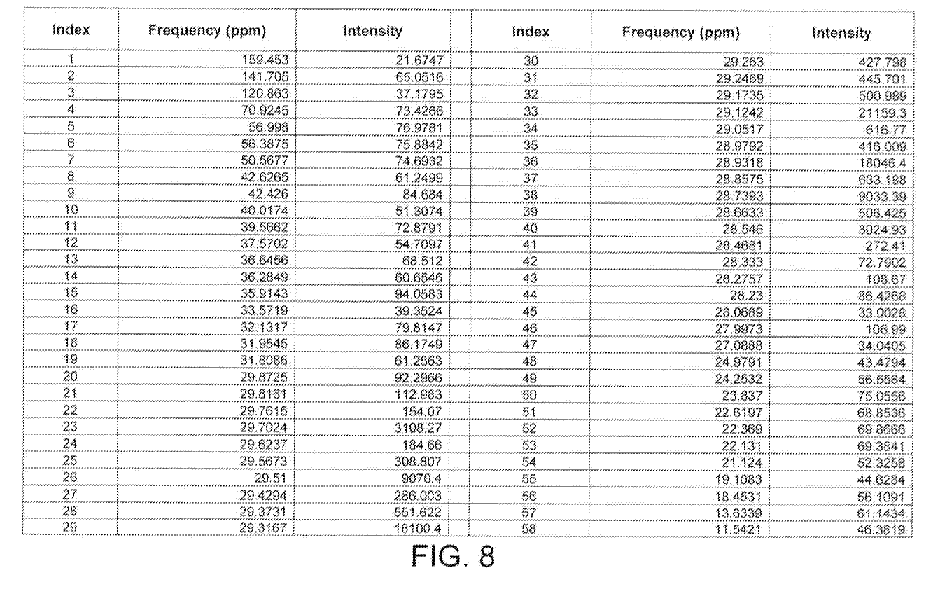

As set forth in FIGS. 3-8, bed bug feces extract exhibit a C-13 NMR peak at δ 159.453 ppm in acetone-d6 solvent. Without being limited by theory, a C-13 NMR diagnostic peak at δ 159.453 ppm may be indicative of, for example, an oxime, hydrazone, imino, amidine, or an imine functional group (See, for example, “The Aldrich Library of ¹³C and ¹H FT NMR Spectra,” Ed. Charles J. Poucher, Volume 3, page 488, 1993 and “Structure Determination of Organic Compounds,” Ed. E. Pretsch, Volume 3, pages 7, 8, and 125, 2000). Moreover, without being limited by theory, the alkyl carbon profile in the C-13 NMR scan correlates well for a ketone oxime in the range from 13 to 40 ppm. In an aspect, without being limited, a compound isolated or identified from insect feces or feces extract according to the methods described herein is 2-heptanone oxime, 2-octanone oxime, 2-hexanone oxime, 4-heptanone oxime, 3-octanone oxime, 3-hexanone oxime, 3-heptanone oxime, 2-heptanone imine and salts thereof, 2-pentanone oxime, 2-pentanone hydrazone, 5-methyl-2-hexanone hydrazone, 2-heptanone O-methoxyimine, 5-methyl-2-hexanone oxime, 2-methyl-3-hexanone oxime, 5-methyl-2-hexene-2-one, cyclohexanone oxime, cyclohexanone hydrazone, cycloheptanone oxime, cycloheptanone hydrazone, 2-Pentanone hydrazone, 3-pentanone hydrazone, 2-hexanone hydrazone, 3-hexanone hydrazone, 2-heptanone hydrazone, 3-heptanone hydrazone, 2-heptanone hydrazone, 3-heptanone hydrazone, 4-heptanone hydrazone, 2-octanone hydrazone, 3-octanone hydrazone, 4-octanone hydrazone, 3-octanone oxime, 2-nonanone hydrazone, 3-nonanone hydrazone, 4-nonanone hydrazone, 5-nonanone hydrazone, 2-nonanone oxime, 3-nonanone oxime, 4-nonanone oxime, 5-nonanone oxime, 2-octanone oxime, 2-propanone O-methyloxime , 1-cyclopropyl-2-heptanone oxime, 1-cyclopropyl-2-heptanone hydrazone, N-(1-Methylpentylidene)methylamine, N-(1-Methylhexyylidene)methylamine, (Z) 2-Hexanone, 3-methyl-, oxime, (E), 2-hexanone, 3-methyl-, oxime, 2-pentanone 3-ethyl-oxime, 2-pentanone, 3,4-dimethyl-oxime, N-methyl pentanimidamide, urea, 1,2,4,5-tetrazine dicarboxamide, or 1,2-Hydrazindicarboxamide, and/or optically active and/or geometric isomers and/or salts thereof.

In an aspect, a compound isolated or identified from insect feces or extract, such as bed bug feces or extract, is a compound of Formula I:

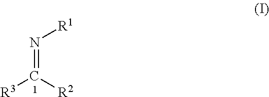

in which,
R¹ represents H, OH, NH₂, substituted or non substituted O—C₁-C₃-alkyl, substituted or non substituted O—C₁-C₆-alkyl, substituted or non substituted C₁-C₆-alkyl, N-methyl, N-ethyl, N-Propyl, N—C₁-C₆-alkyl, N,N-dimethyl,N,N-dialkyl, N—NO₂, N—CN, SO₃H, substituted or non-substituted S(O)—C₁-C₆-alkyl, substituted or non-substituted S—C₁-C₆-alkyl, SH, substituted or non-substituted S(O)₂—C₁-C₆-alkyl, S(O)₂OH, substituted or non-substituted S(O)₂O—C₁-C₆-alkyl, PH₃, P(C₁-C₆-alkyl)₃;
R² and R³ are independently selected from H, methyl, ethyl, propyl, isopropyl, cyclopropyl, butyl, tert-butyl, pentyl, tert-butyl,pentyl, hexyl, heptyl, octyl, nonyl, decyl, OH, COOH, substituted or non substituted C₂-C₁₂-alkyl, substituted or non substituted C₂-C₁₂-alkenyl, substituted or non substituted C₂-C₁₂-alkynyl, substituted or non substituted C₃-C₈-cycloalkyl, allyl, O-allyl, C₁-C₁₂-halogenalkyl, a halogen, tri-fluoromethyl, monohalo, polyhalo, perhaloalkyl, substituted or non substituted C₂-C₁₂-alkenyl, substituted or non substituted C₂-C₆-alkynyl, or where R² and R³ are linked together to form a (CH₂)₃, (CH₂)₄, (CH₂)₅, (CH₂)₆, (CH₂)₇, or a (CH₂)₈ ring structure; and/or
   optically active and/or geometric isomers and/or salts thereof.

In a further aspect, a compound of Formula I is as follows:
R¹ represents H, OH, or NH₂;
R² and R³ are independently selected from H, methyl, ethyl, propyl, isopropyl, butyl, tert-butyl, substituted or non substituted C₂-C₆-alkyl, substituted or non substituted C₂-C₆-alkenyl, a substituted or non substituted C₂-C₆-alkynyl, or a substituted or non substituted C₃-C₆-cycloalkyl; and/or
   optically active and/or geometric isomers and/or salts thereof.

In a further aspect, a compound of Formula I is as follows:
R¹ represents H, OH, or NH₂;
R² and R³ are independently selected from methyl, ethyl, propyl, isopropyl, butyl, tert-butyl, hexyl, heptyl, octyl, nonyl, decyl, and substituted or non substituted C₂-C₆-alkyl; and/or
   optically active and/or geometric isomers thereof.

In a further aspect, a compound of Formula I is as follows:
R¹ represents OH;
R² and R³ are independently selected from methyl, ethyl, propyl, isopropyl, butyl, tert-butyl, hexyl, heptyl, octyl, nonyl, decyl substituted or non substituted C₂-C₆-alkyl; and/or
   optically active and/or geometric isomers and/or salts thereof.

In a further aspect, a compound of Formula I is as follows:
R¹ represents H, OH, or NH₂;
R² is methyl, ethyl, propyl;
R³ is methyl, ethyl, propyl, isopropyl, butyl, tert-butyl, hexyl, heptyl, octyl, nonyl, decyl or substituted or non substituted C₂-C₁₂-alkyl; and/or
   optically active and/or geometric isomers and/or salts thereof.

In a further aspect, a compound of Formula I is as follows:
R¹ represents OH;
R² is methyl;
R³ is butyl, tert-butyl, hexyl, heptyl, octyl, and/or
   optically active and/or geometric isomers and/or salts thereof.

In a further aspect, a compound of Formula I is as follows:
R¹ represents OH;
R² is methyl;
R³ is pentyl, hexyl, heptyl, and/or
   optically active and/or geometric isomers and/or salts thereof.

In an aspect, a compound isolated or identified from insect feces or extract, such as bed bug feces or extract, is a compound of Formula II:

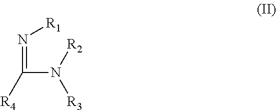

R¹ represents H, NH₂, OH, methyl, ethyl, propyl, isopropyl, butyl, tert-butyl, pentyl, tert-butyl, pentyl, hexyl, heptyl, octyl, nonyl, decyl, substituted or non substituted C₂-C₁₂-alkyl, substituted or non substituted C₂-C₆-alkyl, substituted or non substituted O—C₁-C₃-alkyl, substituted or non substituted O—C₁-C₆-alkyl, N-methyl, N-ethyl, N-Propyl, N—C₁-C₆-alkyl, N,N-dimethyl,N,N-dialkyl, N—NO₂, N—CN, SO₃H, substituted or non-substituted S(O)—C₁-C₆-alkyl, substituted or non-substituted S—C₁-C₆-alkyl, SH, substituted or non-substituted S(O)₂—C₁-C₆-alkyl, S(O)₂OH, substituted or non-substituted S(O)₂O—C₁-C₆-alkyl, PH₃, P(C₁-C₆-alkyl)₃; and
R², R³, and R⁴ are independently selected from H, NH₂, OH, methyl, ethyl, propyl, cyclopropyl, isopropyl, butyl, tert-butyl, pentyl, tert-butyl,pentyl, hexyl, heptyl, octyl, nonyl, decyl, substituted or non substituted C₂-C₁₂-alkyl, substituted or non substituted C₂-C₆-alkyl, substituted or non substituted C₃-C₆-cycloalkyl, substituted or non substituted C₂-C₆-alkenyl, substituted or non substituted C₂-C₆-alkynyl, a halogen, substituted or non substituted O—C₁-C₃-alkyl; and/or
   optically active and/or geometric isomers and/or salts and/or conjugated forms thereof.

In a further aspect, a compound of Formula II is as follows:
R¹ represents H, methyl, ethyl, propyl, isopropyl, butyl, tert-butyl, pentyl, tert-butyl,pentyl, hexyl, heptyl, octyl, nonyl, decyl, substituted or non substituted C₂-C₁₂-alkyl, substituted or non substituted C₂-C₆-alkyl; and
R², R³, and R⁴ are independently selected represents H, methyl, ethyl, propyl, cyclopropyl, isopropyl, butyl, tert-butyl, pentyl, tert-butyl,pentyl, hexyl, heptyl, octyl, nonyl, decyl, substituted or non substituted C₂-C₁₂-alkyl, substituted or non substituted C₂-C₆-alkyl; and/or
   optically active and/or geometric isomers and/or salts thereof.

In a further aspect, a compound of Formula II is as follows:
R¹ represents H, methyl, ethyl, propyl, isopropyl, butyl, tert-butyl, pentyl, tert-butyl,pentyl, hexyl, heptyl, octyl, nonyl, decyl, substituted or non substituted C₂-C₁₂-alkyl, substituted or non substituted C₂-C₆-alkyl; and
R² represents methyl, ethyl, propyl, cyclopropyl, isopropyl, butyl, tert-butyl, pentyl, tert-butyl, pentyl, hexyl, heptyl, octyl, nonyl, decyl, substituted or non substituted C₂-C₁₂-alkyl, substituted or non substituted C₂-C₆-alkyl;
R³ represents H;
R⁴ represents methyl, ethyl, propyl, cyclopropyl, isopropyl, butyl, tert-butyl, pentyl, tert-butyl, pentyl, hexyl, heptyl, octyl, nonyl, decyl, substituted or non substituted C₂-C₁₂-alkyl, substituted or non substituted C₂-C₆-alkyl; and/or
   optically active and/or geometric isomers and/or salts thereof.

In a further aspect, a compound of Formula II is as follows:
R¹ represents H, methyl, or ethyl;
R² represents methyl, or ethyl,
R³ represents H;
R⁴ represents methyl, ethyl, propyl, cyclopropyl, isopropyl, butyl, tert-butyl, pentyl, tert-butyl, pentyl, hexyl, heptyl, octyl, nonyl, decyl, substituted or non substituted C₂-C₁₂-alkyl, substituted or non substituted C₂-C₆-alkyl; and/or
   optically active and/or geometric isomers and/or salts thereof.

In a further aspect, a compound of Formula II is as follows:
R¹ represents H, methyl, or ethyl;
R² represents methyl,
R³ represents H;
R⁴ represents methyl, ethyl, propyl, cyclopropyl, isopropyl, butyl, tert-butyl, pentyl, or a substituted or non substituted C₂-C₆-alkyl; and/or
   optically active and/or geometric isomers and/or salts thereof.

In an aspect, a compound isolated or identified from insect feces or extract, such as bed bug feces or extract, is a compound of Formula III:

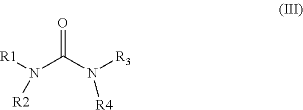

in which
R¹ , R², R³, and R⁴ are independently selected from H, NH₂, OH, methyl, ethyl, propyl, cyclopropyl, isopropyl, butyl, tert-butyl, pentyl, tert-butyl,pentyl, hexyl, heptyl, octyl, nonyl, decyl, substituted or non substituted C₂-C₁₂-alkyl, substituted or non substituted C₂-C₆-alkyl, substituted or non substituted C₃-C₆-cycloalkyl, substituted or non substituted C₂-C₆-alkenyl, substituted or non substituted C₂-C₆-alkynyl, a halogen, substituted or non substituted O—C₁-C₃-alkyl; and/or
   optically active and/or geometric isomers and/or salts thereof.

In a further aspect, a compound of Formula III is as follows: in which
R¹ , R², R³, and R⁴ are independently selected from H, NH₂, OH, methyl, ethyl, propyl, cyclopropyl, isopropyl, butyl, tert-butyl, pentyl, tert-butyl,pentyl, hexyl, heptyl, octyl, nonyl, decyl; and/or
   optically active and/or geometric isomers and/or salts thereof.

In a further aspect, a compound of Formula III is as follows: in which
R¹ and R³ are NH₂; and
R² and R⁴ are independently selected from H, NH₂, OH, methyl, ethyl, propyl, cyclopropyl, isopropyl, butyl, tert-butyl, pentyl, tert-butyl,pentyl, hexyl, heptyl, octyl, nonyl, decyl, substituted or non substituted C₂-C₁₂-alkyl, substituted or non substituted C₂-C₆-alkyl, substituted or non substituted C₃-C₆-cycloalkyl, substituted or non substituted C₂-C₆-alkenyl, substituted or non substituted C₂-C₆-alkynyl, a halogen, substituted or non substituted O—C₁-C₃-alkyl; and/or
   optically active and/or geometric isomers and/or salts thereof.

In a further aspect, a compound of Formula III is as follows: in which
R¹ and R³ are NH₂; and
R² and R⁴ are independently selected from H or substituted or non substituted C₂-C₆-alkyl; and/or
   optically active and/or geometric isomers and/or salts thereof.

In a further aspect, a compound of Formula III is as follows: in which
R¹ and R³ are independently selected from OH and NH₂; and
R² and R⁴ are independently selected from methyl, ethyl, propyl, cyclopropyl, isopropyl, butyl, tert-butyl, pentyl, tert-butyl,pentyl, hexyl, heptyl, octyl, nonyl, decyl; and/or
   optically active and/or geometric isomers and/or salts thereof.

In a further aspect, a compound of Formula III is as follows: in which
R¹ and R³ are NH₂; and
R² and R⁴ are H; and/or
   optically active and/or geometric isomers and/or salts thereof.

In another aspect, a compound of Formula I, II, or III is capable of attracting, aggregating, arresting, controlling, or influencing the behavior of an insect, such as a bed bug.

Any solvent capable of dissolving the insect feces, such as an alcohol or halogenated carbon solvents, are encompassed by the disclosure. In an aspect, the solvent used to dissolve the insect feces is an alcohol. In another aspect, the solvent is methanol, ethanol, methylene chloride, chloroform, or acetone.

In an aspect, the insect feces is collected from an insect after about 1 to about 10 days, about 1 to 5 days, or about 1, 2, 3, 4, 5 days of feeding. In another aspect, insect feces is collected by placing a bed bug on paper bedding and collecting the feces. In another aspect, additional support material according to the disclosure may be used as insect bedding. In yet another aspect, an insect is placed on a support material bedding for about 1 to about 10 days, about 3 to about 8 days, about 4 to about 6 days, or until such a time that sufficient feces is collected to analyze the feces sample.

In another aspect, the disclosure provides for a compound, composition, device, support material, kit, as well a method of attracting, detecting, eradicating, controlling, regulating or influencing the behavior of and/or killing an insect by utilizing an insect compound identified or isolated from insect feces as described herein.

The disclosure also provides for a detection device for monitoring or detecting bed bug infestation, comprising
a first plate and a second plate, wherein the first plate and the second plate are separated by an internal spacing sized to permit entry into the detection device by a bed bug; and
a composition comprising an insect attractant, arresting, and/or aggregation compound identified or isolated from insect feces.

In an aspect, the disclosure provides for a support material combined with a compound or composition described herein. In another aspect, the support material can be coated or impregnated with an insect attractant, arresting, and/or aggregation compound or composition described herein. An insecticide can also be added to the support material coated or impregnated with an attractant, arresting, and/or aggregation compound or composition described herein. In another aspect, the composition, device, or support material does not include an insecticide. Examples of support materials capable of being used with compounds described herein include an absorbent material, wood, cardboard, corrugated cardboard, cotton, wallpaper, paper, plastic, a plastic tube, and metal. Support materials with a low heat transfer rate may also be suitable.

In another aspect, the support material combined with an attractant, arresting, and/or aggregation compound or composition described herein can be used to monitor or attract insects, such as bed bugs. In one aspect, the support material is combined with an attractant, arresting, and/or aggregation compound described herein and an insecticide. Such a support material can be used in a method of eradicating or killing insects. The support material can also be used in conjunction with a device, such as an insect trap or insect detection device or monitor. Without being limited, the detection devices described herein can be used with the support material. In another aspect, the insect trap, detection device, or monitor is specific for bed bugs.

In another aspect, the disclosure also provides for a method of luring or arresting an insect into an area treated with an insect attractant, arresting, and/or aggregation compound identified or isolated from insect feces as described herein. In an aspect, an insect, such as a bed bug, is arrested in a given area for about 10 minutes, about 30 minutes, about 1 hour, about 8 hours, about 24 hours, about 2 days, about 7 days, about 2 weeks, or about 1 month. In one aspect, a support material is present in the treated area. In another aspect, an insect, such as a bed bug, is arrested in a given area for from about 1 hour to about 8 hours, from about 12 hour to about 2 days, or from about 1 day to about 7 days.

In one aspect, a support material is present in the treated area. In another aspect, the support material is capable of being coated, impregnated, or sprayed with a compound described herein. Without being limited, the support material can be an absorbent material, wood, cardboard, corrugated cardboard, cotton, wallpaper, paper, plastic, a plastic tube, or metal. In an aspect, the treated area can be, for example, a bed, a pillow, a pillow case, a mattress, a box spring, a bed frame, a headboard, a sheet, a carpet, furniture, a picture frame, a book case, an upholstered chair, a sofa and wood furniture, along and beneath a baseboard, a floor area, under a bed and a couch or any place a bed bug is prone to rest or hide.

In accordance with a further aspect, there is provided a method for monitoring the presence of insects, such as bed bugs, in multiple discreet areas such as in hotel rooms, bedrooms, animal holding areas such as chicken houses, barn stalls, etc. In accordance with this method, a device which allows for the entry of an insect such as bed bug may be placed in each location sought to be monitored. There can be one device or hundreds or more devices depending on how many discreet areas need to be monitored separately. For example, in a hotel, a separate device could be mounted to each bed in each room in the hotel. The device can be any desired device, for example, the device as disclosed herein. In an aspect, the device or devices can be checked on a periodic basis, for example, weekly, biweekly, every 24 hours, or as desired, and if the device has evidence of insect contact such as by viewing the insect in the device, by noticing feces in the device and/or by presence of blood from insect feces, then the area where that particular device was mounted is then subjected to a treatment regime to eliminate or reduce the activity of the insect in that area.

In another aspect, the disclosure provides for a method of treating, controlling, monitoring, or detecting bed bug infestation comprising adding an insect attractant, arresting, and/or aggregation compound identified or isolated from insect feces as described herein to a support material, detection device, or trap and placing the support material, device, or trap in an area prone to bed bug infestation. In another aspect, the support material, detection device, or trap is placed in an area after identification of bed bug infestation. In yet another aspect, the support material, detection device, or trap is placed in an area prior to identification of bed bug infestation. In another aspect, the disclosure provides a method of killing or eradicating bed bugs comprising adding an attractant, arresting, and/or aggregation compound or composition described herein to a support material, detection device, or trap and placing the support material, device, or trap in an area prone to bed bug infestation.

In an aspect, the area prone to bed bug infestation includes, for example, a bed, a pillow, a pillow case, a mattress, a box spring, a bed frame, a headboard, a sheet, a carpet, furniture, a picture frame, a book case, an upholstered chair, a sofa and wood furniture, along and beneath a baseboard, a floor area, under a bed and a couch or any place a bed bug is prone to rest or hide. In another aspect, from about 1 to about 6, from about 2 to about 4, or from about 1, 2, 3, 4, 5, 6, 7, 8, 9, or 10 or more detection devices, traps, or support materials are placed in an area prone to bed bug infestation. In yet another aspect, the device, trap, or support material is evaluated by an individual or PMP for bed bug infestation from about 1 to about 5 times, about 1 to about 3 times, or from about 2 to about 4 times a day, from about 1 to about 10 times, from about 3 to about 8 times, or from about 1 to about 5 times a week, or from about 1 to about 30 times, from about 5 to about 20 times, from about 1 to about 5 times a month.

In an aspect according to the disclosure, the insect is a bed bug. In another aspect, the bed bug is from the species Cimex lectularius, Cimex hemipterus, Leptocimex boueti, Cimex pilosellus, Cimex pipistrella, or Haematosiphon inodora. In yet another aspect, the bed bug is from Cimex sp. or other genera in the Cimicidae family, for example the genus Haematosiphon, or the genus Oeciacus. In a further aspect, the bed bug is a first, second, third, fourth, or fifth instar. In another aspect, the bed bug is a fourth or fifth instar bed bug. In a further aspect, the bed bug is a large nymph bed bug.

In an aspect, the methods, compositions, and devices may be practiced with additional insects, such as Blood-feeding Reduvidae, for example a kissing bug, or other Heteroptera that aggregate during all or part of their life cycle. In another aspect, the methods, compositions, and devices may be practiced with additional insects, such as mosquitoes, lice, ticks, fleas, deer ticks, poultry lice or other arthropods such as tick species. In yet another aspect, the methods, compositions, and devices may be practiced with additional arthropods or blood feeding animals or insects. In an aspect, the behavior of the above groups may be altered by common chemical or biological compounds. In yet another aspect, the above groups are attracted, arrested, or aggregated by compounds, such as the ones described herein.

### Compositions:

The disclosure provides for bait compositions comprising an insect attractant, arresting, and/or aggregation compound identified or isolated from insect feces. In an aspect, the disclosure provides for a method of treating insect infestation by providing a bait material comprising an insect attractant, arresting, and/or aggregation compound identified or isolated from insect feces. The bait composition may be used in any of the methods described herein. Without being limited, specific compounds capable of being used together with insect attractant, arresting, and/or aggregation compounds as described herein, include one or more, two or more, three or more, or four or more of 2-heptanone oxime, 2-octanone oxime, 2-hexanone oxime, 4-heptanone oxime, 3-octanone oxime, 3-hexanone oxime, 3-heptanone oxime, 2-heptanone imine and salts thereof, 2-pentanone oxime, 2-pentanone hydrazone, 5-methyl-2-hexanone hydrazone, 2-heptanone 0-methoxyimine, 5-methyl-2-hexanone oxime, 2-methyl-3-hexanone oxime, 5-methyl-2-hexene-2-one, cyclohexanone oxime, cyclohexanone hydrazone, cycloheptanone oxime, cycloheptanone hydrazone, 2-Pentanone hydrazone, 3-pentanone hydrazone, 2-hexanone hydrazone, 3-hexanone hydrazone, 2-heptanone hydrazone, 3-heptanone hydrazone, 2-heptanone hydrazone, 3-heptanone hydrazone, 4-heptanone hydrazone, 2-octanone hydrazone, 3-octanone hydrazone, 4-octanone hydrazone, 3-octanone oxime, 2-nonanone hydrazone, 3-nonanone hydrazone, 4-nonanone hydrazone, 5-nonanone hydrazone, 2- nonanone oxime, 3-nonanone oxime, 4-nonanone oxime, 5-nonanone oxime, 2-octanone oxime, 2-propanone O-methyloxime, 1-cyclopropyl-2-heptanone oxime, 1-cyclopropyl-2-heptanone hydrazone, N-(1-Methylpentylidene)methylamine, N-(1-Methylhexyylidene)methylamine, (Z) 2-Hexanone, 3-methyl-, oxime, (E), 2-hexanone, 3-methyl-, oxime, 2-pentanone 3-ethyl-oxime, 2-pentanone, 3,4-dimethyl-oxime, N-methyl pentanimidamide, urea, 1,2,4,5-tetrazine dicarboxamide, or 1,2-Hydrazindicarboxamide, and/or optically active and/or geometric isomers and/or salts thereof.

In an aspect, the disclosure provides for a composition comprising a compound described herein together with one or more compounds selected from cholesterol, (E)-2-hexenal, (E)-2-hexenol, (E,E)-2,4-hexadienal, (E)-2-octenal, or 2-Heptanone.

The disclosure also provides for compositions comprising, consisting of, or consisting essentially of an insect attractant compound described herein together with an insecticide. Any insecticide could be used in connection with the present compositions and methods. Notably, for example, any insecticide capable of killing an insect, such as a bed bug, can be combined with insect attractant compounds described herein. Examples of insecticides which may optionally be admixed include: phosphoric esters, such as acephate, chlorpyrifos, dichlorovos, malathion or propetamphos; carbamates, such as bendiocarb, carbaryl, or propoxur; pyrethroids, such as allethrin, bifenthrin, bioallethrin, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, esfenvalerate, etofenprox, fenpropathrin, fenvalerate, flumethrin, tau-fluvalinate, halfenprox, imiprothrin, permethrin, phenothrin, prallethrin, pyrethrins (pyrethrum), resmethrin, silafluofen, tefluthrin, tetramethrin or transfluthrin; nitroimines and nitromethylenes, such as 1-[(6-chloro-3-pyridinyl)-methyl]-4,5-dihydro-N-nitro-1H-imidazol-2-amine (imidacloprid), N-R6-chloro-3-pyridyl)methyldN.sup.2-cyano-N.sup.1-methylacetamide (NI-25); phenylpyrazoles, such as fipronil or ethiprole; avermectins, such as abamectin or emamectin benzoate; spinosyns, such as spinosad or spinetoram; oxadiazines, such as indoxacarb or metaflumizone; anthranilic diamides, such as flubendiamid, chlorantraniliprole or cyantraniliprole; sulfoximines, such as sulfoxaflor; insect growth regulators, such as methoprene, chlorfluazuron, flufenoxuron, pyriproxyfen, triflumuron or flufenoxuron; and other insecticidally active compounds, such as azadirachtin, chlorfenapyr, hydramethylnon, petroleum oils or botanical oils. Any of the above insecticide compositions can be used in conjunction with the insect attractant, arresting, and/or aggregation compounds and compositions described herein.

In addition, compositions of the present disclosure and methods for their use can include other actives and inactives as well known in the art

In another aspect, a attractant, arresting, and/or aggregation compounds described herein may be combined with other compounds present in bed bug extract. Moreover, additional compounds isolated from bed bugs can be combined with attractant, arresting, and/or aggregation compounds described herein. Many such compounds are representative of aggregation, alarm, and/or sexual pheromones. In an aspect, other compounds or compositions capable of being combined with attractant, arresting, and/or aggregation compounds described herein include those disclosed in “Identification of the Airborne Aggregation Pheromone of the Common Bed Bug, Cimex lectularius”, Journal of Chemical Ecology, vol 34, no. 6, June 2008, which is incorporated herein by reference in its entirety. Other suitable attractants or arrestants include food attractants in any form, such as in solid, gel or liquid form. It is also possible to utilize carbon dioxide, heat, methanol, methane, furan, pyridine, human perspiration, lactic acid, butyric acid, octenol, trans-2-octenal, trans-2-hexenal, 2-butanone, lactic acid, indole, 6-methyl-5-hepten-2-one, geranyl acetone, 1-dodecanol, 3-methyl-l-butanol, carboxylic acids, urea, and sebum (a component of skin oil). Suitable attractants or arrestants also can include one or more harboraging agents. Any of the above compounds or compositions attractants or arrestants may also be used in conjunction with compounds or compositions described herein.

Compounds and compositions of the disclosure may be present in any amount sufficient to attract, arrest, aggregate, control, an insect, such as a bed bug. In an aspect, the disclosure provides for a composition comprising from about 200 ng to about 10 mg of a compound described herein. In another aspect, a compound described herein can be present in an amount from about 500 ng to about 5 mg, from about 500 ng to about 2 mg, from about 500 ng to about 1 mg, from about 1000 ng to about 1 mg, from about 0.01 mg to about 5 mg, from about 0.01 mg to about 1 mg, from about 0.1 mg to about 5 mg, from about 0.1 mg to about 1 mg; from about 0.5 mg to about 2 mg, and from about 0.5 mg to about 1 mg. In another aspect, a compound described herein can be present in an amount from about 100 ng to about 5000 ng, from about 100 ng to about 1000 ng, and from about 100 ng to about 500 ng.

In an aspect, the disclosure provides for a composition comprising from about 1 μg to about 1000 μg of a compound described herein. In another aspect, a compound described herein can be present in an amount from about 1 μg to about 500 μg, from about 10 μg to about 300 μg, from about 10 μg to about 100 μg, or from about 20 μg to about 80 μg.

In another aspect, a compound described herein can be present from about 10 ng, about 50 ng, about 100 ng, about 200 ng, about 500 ng, about 1000 ng, about 5000 ng, about 0.01 mg, about 0.2 mg, about 0.5 mg, about 1 mg, about 2 mg, about 3 mg, about 5 mg about 10 mg, about 20 mg, or about 50 mg. In yet another aspect, a compound described herein can be present from about 10 ng or more, about 50 ng or more, about 100 ng or more, about 200 ng or more, about 500 ng or more, about 1000 ng or more, about 5000 ng or more, about 0.01 mg or more, about 0.2 mg or more, about 0.5 mg or more, about 1 mg or more, about 2 mg or more, about 3 mg or more, about 5 mg or more, about 10 mg or more, about 20 mg or more, or about 50 mg or more.

In another aspect, a composition of the disclosure can contain from about 0.1% to about 99.9% by weight of a compound according to the disclosure. In another aspect, a composition of the disclosure can contain from about 10% to about 90%, from about 20% to about 80%, from about 25% to about 75%, from about 30% to about 70%, or from about 40% to about 60% by weight of an insecticide or compound according to the disclosure.

In yet another aspect, a composition of the disclosure can contain from about 10%, about 20%, about 30%, about 40%, about 50%, about 60%, about 70%, about 80%, or about 90% by weight of a compound or insecticide according to the disclosure. Compositions of the disclosure can also contain from about 10% or more, about 20% or more, about 30% or more, about 40% or more, about 50% or more, about 60% or more, about 70% or more, about 80% or more, or about 90% or more by weight of a compound or insecticide according to the disclosure by weight of an insecticide or insect attractant according to the disclosure.

Compositions according to the disclosure may also include additional components such as excipients, binders, diluents, pharmaceutical carriers, and adjuvants. In an aspect, compositions according to the disclosure can include diatomaceous earth and/or silica gel, such as Dri-die, as described in Benoit et at. (Journal of Medical Entomology 46(3):572-579. 2009), which is herein incorporated by reference in its entirety. Plastizers such as dioctyl adipate and bis-2-ethyl-hexyl adipate, can also be added to the compositions.

In a non-limiting aspect, compositions according to the disclosure may be formulated and used in various forms, such as aerosol dispenser, bait (ready for use), bait concentrate, block bait, capsule suspension, cold fogging concentrate, dustable powder, emulsifiable concentrate, emulsion oil in water, emulsion water in oil encapsulated granule, fine granule, grain bait, granular bait, granule, macrogranule, microgranule, oil dispersible powder, oil miscible flowable concentrate, oil miscible liquid, paste, plate bait, scrap bait, smoke candle, smoke cartridge, smoke generator, smoke pellet, smoke rodlet, smoke tablet, smoke tin, soluble concentrate, soluble powder, suspension concentrate, oil dispersible or suspension in oil, tracking powder, ultra low volume (ulv) liquid, ultra low volume (ulv) suspension, vapour releasing product, water dispersible granules or tablets, water dispersible powder for slurry treatment, water soluble granules or tablets, or water soluble powder for seed treatment and wettable powder or any other forms of slow release/controlled release formulations.

### Detection Device:

In another aspect, the disclosure provides for a detection device including a compound or composition as described herein.

A method for detecting the presence of bed bugs using devices and compounds described herein is also provided. The method allows for the detection of bed bugs even when there are no bed bugs visible at the time of evaluation of the detection. This method takes advantage of the fact that bed bugs' feed on blood and those remainders of the blood such as heme, globin, or hemeoglobin can be detected in the bugs' feces. Another aspect of this method comprises applying a fecal occult blood test to insect feces to detect the presence of blood, which is a strong indication of bed bugs as the source of the feces. Generally, any test system can be used, for example, a detection system which allows for a visual readout, e.g., a color reaction.

In order to be able to easily apply this test to bed bug feces, it is useful to combine this method with the devices of the present disclosure which may be entered by bed bugs, and/or used by bed bugs for resting, and thus serve as a location for releasing feces. The feces can then be easily checked for the presence of blood with the above method.

In an aspect, a method for detecting the presence of bed bugs is also provided, comprising: providing a detection device containing a compound or composition described herein, and wherein the detection device contains an internal volume and an opening sized to permit entry into the detection device by one or more bed bugs; and detecting the presence of the bed bugs with a fecal occult blood test performed either in the device or on content of the device after removal of the same from the device.

In yet another aspect, the device described above may be prepared in such a way that a test for the presence of fecal blood can easily be performed and evaluated. To achieve this, in an aspect, the detection device contains an absorbent layer impregnated with a fecal blood detection agent, for example, a paper layer impregnated with a fecal blood detection agent. In an aspect, the absorbent layer impregnated with fecal blood is attached to a first plate. One example of a fecal blood detection agent is a guaiac-resin, which is capable of displaying visible indicia in presence of fecal blood from the bed bug when hydrogen peroxide as developing agent is added. In another aspect, in order to allow a read-out within the device, a plate of the device is transparent.

An example of a suitable detection device for use with compounds and compositions of the disclosure may be found in U.S. patent application Ser. No. 12/392,417, the contents of which are herein incorporated by reference in their entirety. Moreover, in an aspect, a detection device for monitoring bed bug infestation is provided, comprising: a first plate and a second plate, wherein the first plate and the second plate are separated by an internal spacing sized to permit entry into the detection device by a bed bug; a support structure between the first plate and the second plate; and a compound or composition as described herein. In an aspect, the attractant, arresting, and/or aggregation compound or composition described herein is present on or in an absorbent layer. In another aspect, the absorbent layer is fixed to the first plate. The support structure between the first and second plate can be positioned in any desired manner and for any desired purpose such as for maintaining the internal spacing and/or to provoke thigmotactic behavior of the bed bugs.

Bed bugs are attracted to materials with a rough surface texture and surface porosity. Examples of materials that may be used to form the detection device itself and/or be incorporated into the detection device include wood, cardboard, corrugated cardboard, cotton, or wallpaper. Materials with a low heat transfer rate may also be suitable in some cases.

In an aspect, if insects such as bed bugs are detected in one of the areas being monitored, bed linens and clothing can be first removed from the area. Beds can than be disassembled and bed bug-infested mattresses, box springs, couches or other belongings can be discarded. The area can than be treated attractant, arresting, and/or aggregation compounds and compositions according to the disclosure in conjunction with any insecticide known to be active against the insect that has infested the area.

In an aspect, after treatment, additional follow-up inspections can be made, for example, on a biweekly basis. Since bed bugs are often hard to eradicate, to achieve the desired outcome, thorough inspections and repeated applications should be made and a sufficient quantity of insecticide should be used. The amount of time spent in each infested area on the initial treatment can range from 1 to 2 hours, with each follow-up service or treatment can last at least an additional hour or more. Once an area has been treated, a new device can be installed in that area for future monitoring of the presence of insects.

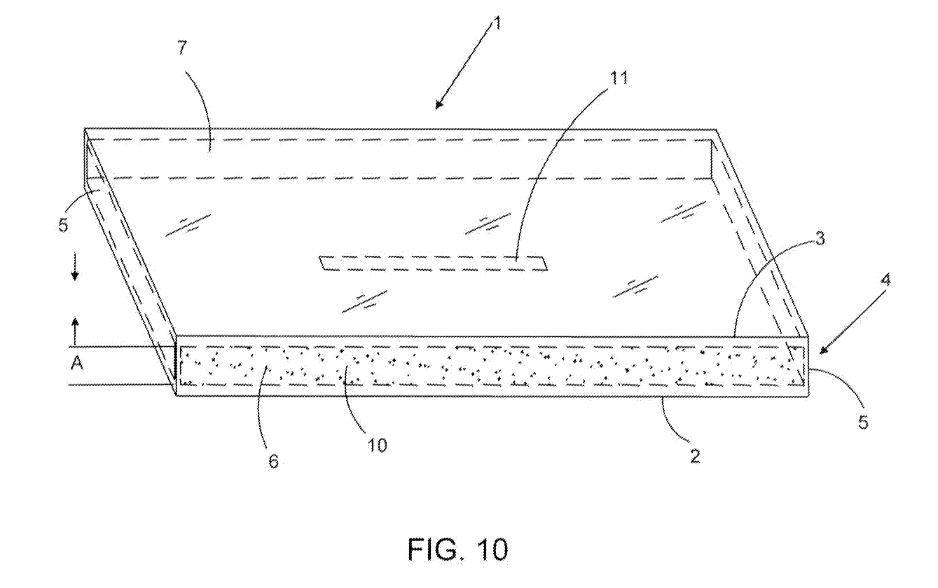

FIG. 10 describes a detection device 1 for monitoring bed bug infestation according to one embodiment. As shown in FIG. 10, the detection device 1 includes a first plate 2 and a second plate 3. The first plate 2 is a surface over which the bed bugs are expected to traverse. The first plate 2 and the second plate 3 are separated by an internal spacing A, sized to permit entry into the detection device 1 by one or more bed bugs (not shown). A support structure 4 exists between the first plate 2 and the second plate 3 for maintaining the internal spacing A. Such support structure 4 can simply be side walls 5 at opposing ends of the device 1, such that entry slots 6, 7 are defined. In an aspect, the attractant, arresting, and/or aggregation compound or composition described herein can be used with the detection device of FIG. 10.

### Kit:

Also provided is a kit comprising, consisting of, or consisting essentially of a compound or composition as described herein. In one aspect, the kit comprises an insect attractant, arresting, and/or aggregation compound identified from insect feces or fecal extract using one or more analytical techniques, for example, gas chromatography, nuclear magnetic resonance (NMR), Carbon-13 NMR, mass spectroscopy, or high performance liquid chromatography (HPLC).

In another aspect, the kit comprises a composition that includes a compound identified or isolated from insect feces or fecal extract, wherein the compound exhibits a peak at about δ 159.453 ppm when analyzed by Carbon-13 NMR.

In yet another aspect, the kit comprises a compound identified or isolated from insect feces or fecal extract, wherein the compound exhibits a peak at about δ 159.453 ppm when analyzed by Carbon-13 NMR and comprises an oxime, a hydrazone, amidine, or an imine functional group. In an aspect, the kit comprises an insect attractant, arresting, and/or aggregation compound identified or isolated from insect feces or fecal extract, wherein the insect attractant exhibits a peak at about δ 159.453 ppm when analyzed by Carbon-13 NMR and comprises from about six to ten carbon atoms and an oxime group. In yet another aspect, the kit comprises a composition including 2-heptanone oxime, 2-octanone oxime, 2-hexanone oxime, 4-heptanone oxime, 3-octanone oxime, 3-hexanone oxime, 3-heptanone oxime, 2-heptanone imine and salts thereof, 2-pentanone oxime, 2-pentanone hydrazone, 5-methyl-2-hexanone hydrazone, 2-heptanone O-methoxyimine, 5-methyl-2-hexanone oxime, 2-methyl-3-hexanone oxime, 5-methyl-2-hexene-2-one, cyclohexanone oxime, cyclohexanone hydrazone, cycloheptanone oxime, cycloheptanone hydrazone, 2-Pentanone hydrazone, 3-pentanone hydrazone, 2-hexanone hydrazone, 3-hexanone hydrazone, 2-heptanone hydrazone, 3-heptanone hydrazone, 2-heptanone hydrazone, 3-heptanone hydrazone, 4-heptanone hydrazone, 2-octanone hydrazone, 3-octanone hydrazone, 4-octanone hydrazone, 3-octanone oxime, 2- nonanone hydrazone, 3- nonanone hydrazone, 4-nonanone hydrazone, 5- nonanone hydrazone, 2- nonanone oxime, 3- nonanone oxime, 4-nonanone oxime, 5- nonanone oxime, 2-octanone oxime, 2-propanone O-methyloxime , 1-cyclopropyl-2-heptanone oxime, 1-cyclopropyl-2-heptanone hydrazone, N-(1-Methylpentylidene)methylamine, N-(1-Methylhexyylidene)methylamine, (Z) 2-Hexanone, 3-methyl-, oxime, (E), 2-hexanone, 3-methyl-, oxime, 2-pentanone 3-ethyl-oxime, 2-pentanone, 3,4-dimethyl-oxime, N-methyl pentanimidamide, urea, 1,2,4,5-tetrazine dicarboxamide, 1,2-Hydrazindicarboxamide, and/or optically active and/or geometric isomers and/or salts thereof.

The kit may also include instructions for describing procedures associated with the disclosed methods. In yet another aspect, the kit comprises instructions for using a compound or composition as described herein for attracting, arresting, aggregating, detecting, controlling, eradicating, or killing an insect. In an aspect, the kit is configured to treat, detect, or control bed bug infestation.

In another aspect, the kit comprises an insect detection device, support material, bait, or trap as described herein together with a compound or composition as described herein. The kit may also include an insecticide or other composition component as described herein. In yet another aspect, the kit does not include an insecticide.

In another aspect, the kit can contain a single-use disposable detection device, bait, support material, or trap. The kit may also contain a reusable multi-use detection device, bait, support material, or trap.

In another aspect, the kit may include a supplement to the detection device, bait, support material, or trap. For example, the supplemental kit may contain an additional bait composition or other supplement to the to the detection device, bait, support material, or trap. In one aspect, the supplemental kit is packaged separate from the detection device, bait, support material, or trap. In another aspect, the supplemental kit is packaged together with the detection device, bait, support material, or trap.

### EXAMPLES

### Example 1

The following represents an example large nymph bed bug bedding and feces extraction procedure and analysis.

Approximately 1-4 days post feeding, 150-200 large nymph bed bugs (late instar) are placed on filter paper bedding. The large nymphs are left for approximately 5-7 days on filter paper bedding. This allows for ample feces to build up. After this time period, the large nymphs are removed from the filter paper bedding. The filter paper bedding is cut into strips and placed into a glass column. In an aspect, the entire filter paper bedding is used (i.e. both sections of the bedding for the extraction).

Approximately 5 ml of methanol is measured in a graduated cylinder and poured into the glass column (over the strips). The resulting extract material is collected in a vial. In an aspect, the glass rod is used to gently squeeze out extra liquid. The extract material is poured over the strips about 15-20 times, alternating between the vial and a beaker to collect the extract. In an aspect, the resulting liquid material exhibits a yellowish—light brown color. In another aspect, approximately 4 ml of liquid is recovered.

The recovered extract can be tested with detection devices or traps. The recovered extract material is also used to test the behavior response of bed bugs and is tested by the two-choice test of Example 2.

### Example 2

In this Example, bed bugs were tested in a two-choice bioassay test using the following procedure:

Individual behavioral responses to test solutions were carried out in 500-ml glass beakers. The bottom-inside surfaces of the 500-ml glass beakers were covered with a white filter paper (70 mm diameter; Fisherbrand, quality P4). The paper was fixed to the glass with double-sided tape. After each assay, papers were changed and beakers were rinsed with acetone. The choices in assays consisted of tents made of filter paper (15×12 mm, Whatman no. 2) folded in the middle to offer a tent-like shelter of 15 mm x 6 mm with two open ends. The assays lasted about 16 h (from about 4:30 p.m. to 8:30 a.m of the next day) with the following light-dark regimen: lights off at 9 p.m. and lights on at 7 a.m. (the same light cycle used during rearing). The room temperature remained at 24±2° C. Prior to the releases, insects were acclimated to the environment for 15 min in a shell vial (21 diam.×70 mm height) which was placed inverted in the center of the arena. At the end of the test, the location of the insects resting in a tent or wandering in the arena (considered as “out”) was recorded. The number of responses was further analyzed by a binomial test with exact two-tailed P values, with the null hypothesis that the tent were chosen with equal probability.

### Example 3

In this example, bed bug feces extract was tested by flash chromatography and gas chromatography.

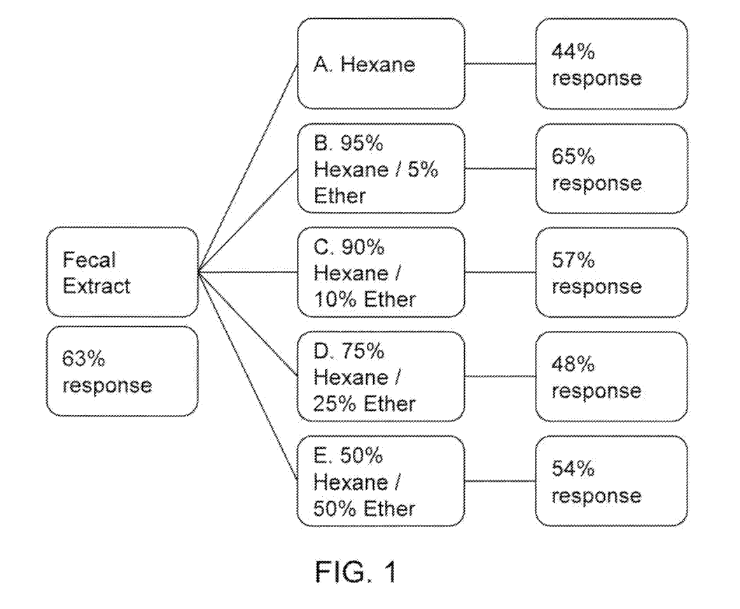

5,000 nymphal bed bug feces equivalents were extracted in methanol. One nymphal equivalent represents the defecation products of one bed bug nymph over the course of 4 days. The volume of the methanol extract was reduced under nitrogen to a concentration of 200 equivalents per ml. A 2 ml aliquot of the pooled extract was added to 0.2 g of florisil to prepare a slurry. The slurry was dried under nitrogen. Florisil residue was then added to the top of a column of 0.2 g of clean florisil. A series of solutions of increasing polarity (hexane, hexane with 5% ether, hexane with 10% ether, etc.) was passed through the column with eluted fractions collected. The original pooled extract and each of the fractions were evaluated by the two-choice bioassay test. In each assay the response to the fraction was compared to the response to the appropriate solvent mix. As set forth in FIG. 1, the original extract and the 95% Hexane/5% Ether fraction (fraction B) stimulated aggregation (i.e., the response was significantly greater than 50%). The expected response would be 50% if no stimulus is present.

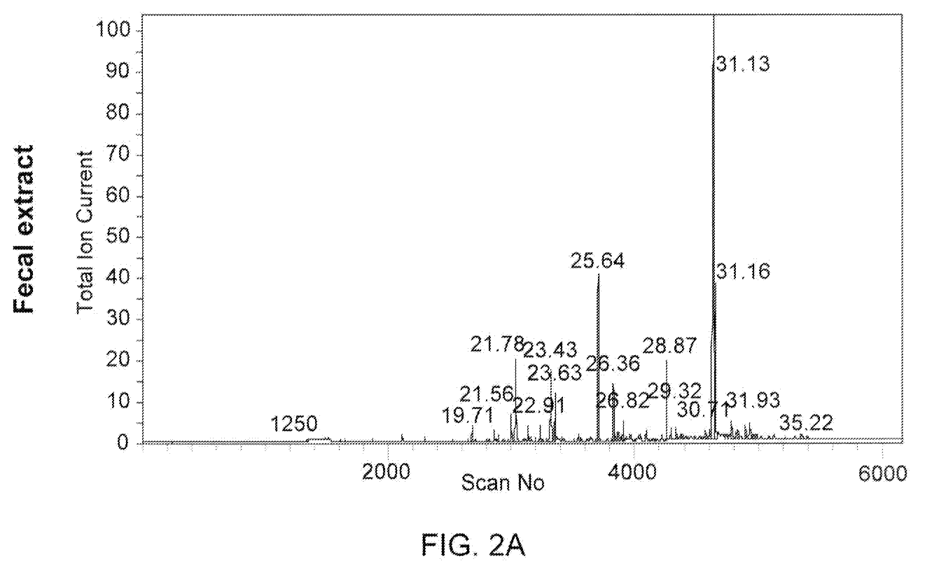

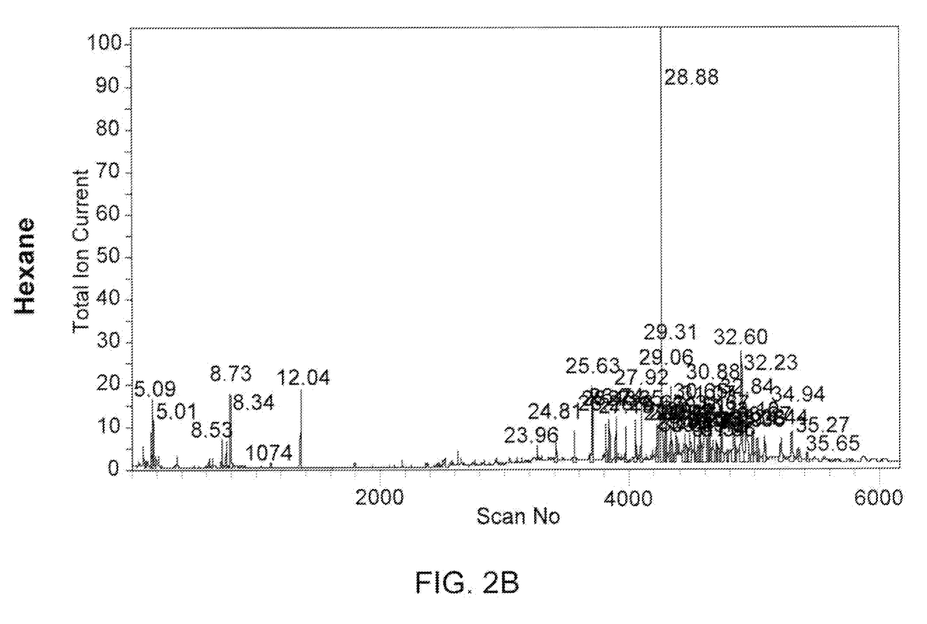

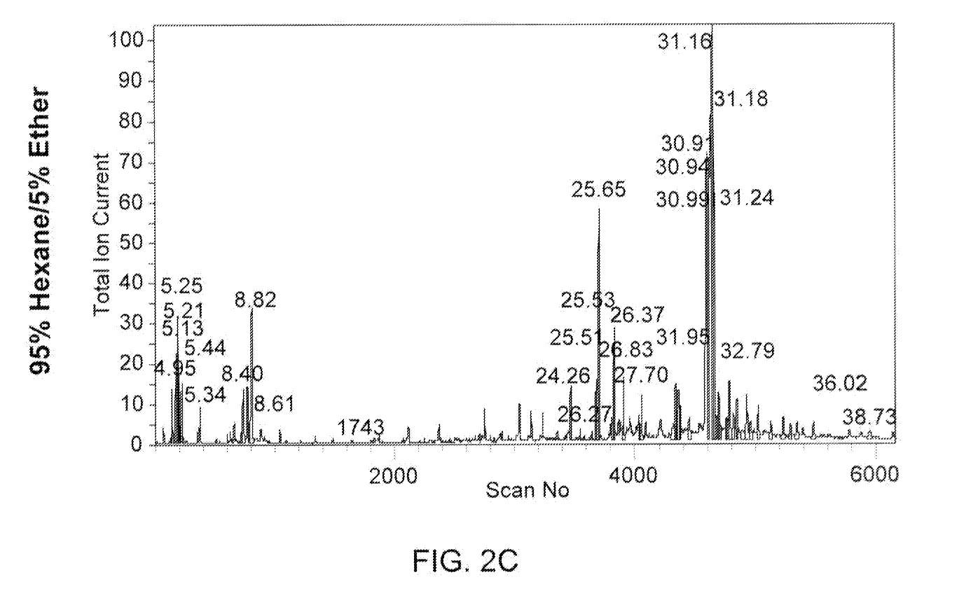

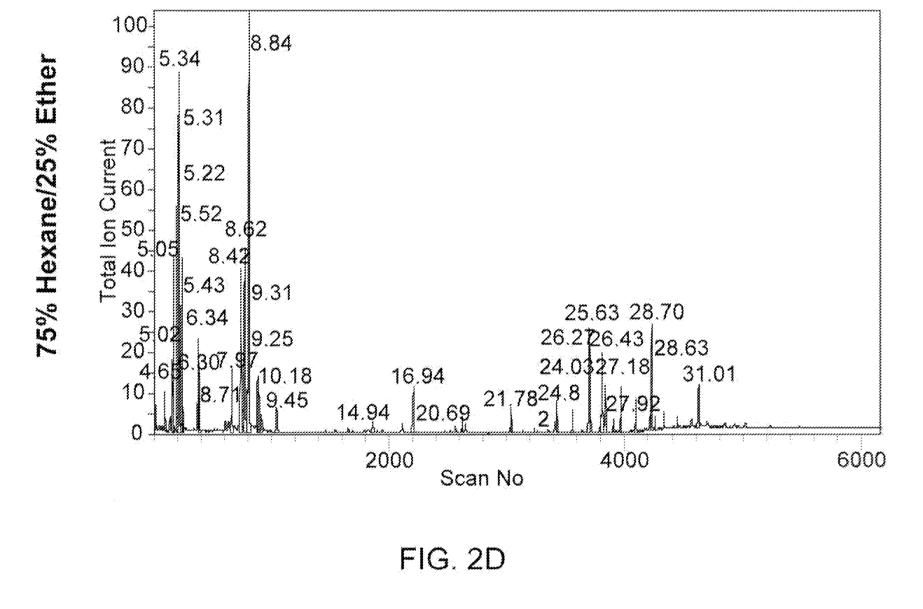

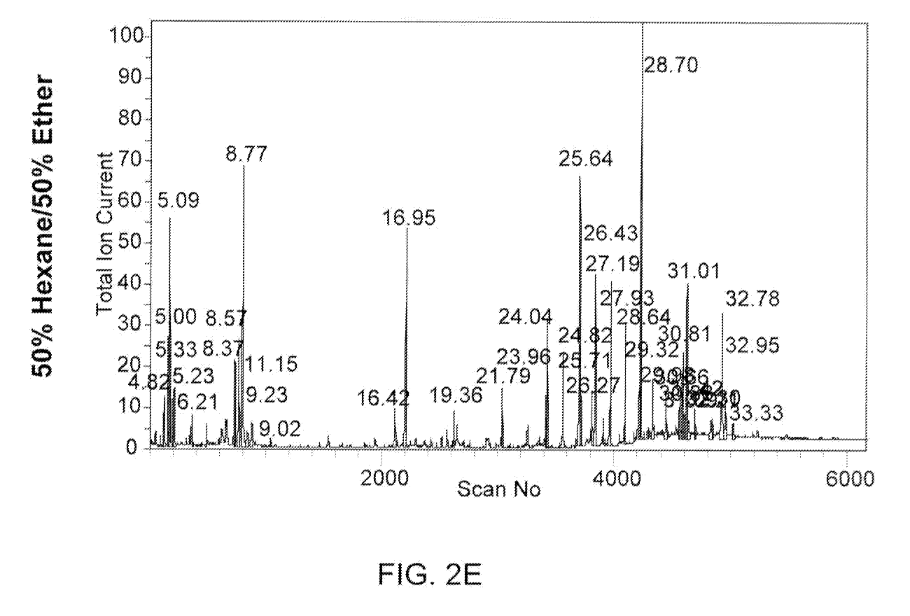

A Gas Chromatography profile is set forth in FIG. 2 for each extract fraction as well as the original extract material. As set forth in FIG. 2, the gas chromatography profiles of the original extract; hexane; 95% hexane/5% ether; 75% hexane/25% ether; and 50% hexane/50% ether were tested.

### Example 4

In this example, bed bug feces extract obtained from the methodology of Example 1 was evaluated by C-13 NMR. The C-13 NMR scans are set forth in FIGS. 3-8.

A bed bug feces extract dissolved in methanol was evaporated in a fume hood at room temperature for two days. Acetone-d6 was added to the resulting residue. The mixture was not completely dissolved. The resulting solution was placed in a NMR tube and C-13 NMR scans were taken. The C-13 NMR scan was also taken for cholesterol (Aldrich) for a side-by-side spectral comparison

The resulting peaks with a match to cholesterol were discounted. Peaks that appeared significant but not matched with cholesterol were present at δ 13.635, 22.620, 24.979, 27.089, 31.809, 33.575 and 159.453 ppm. Without being limited by theory, a C-13 NMR diagnostic peak at δ 159.453 ppm may be indicative of, for example, an oxime, hydrazone, imino, amidine, or imine functional group.

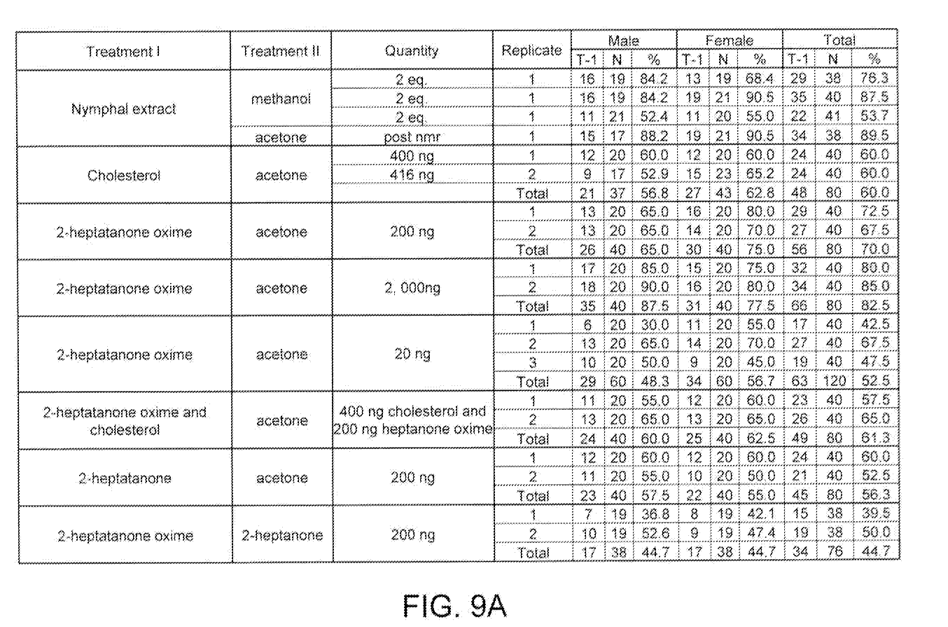

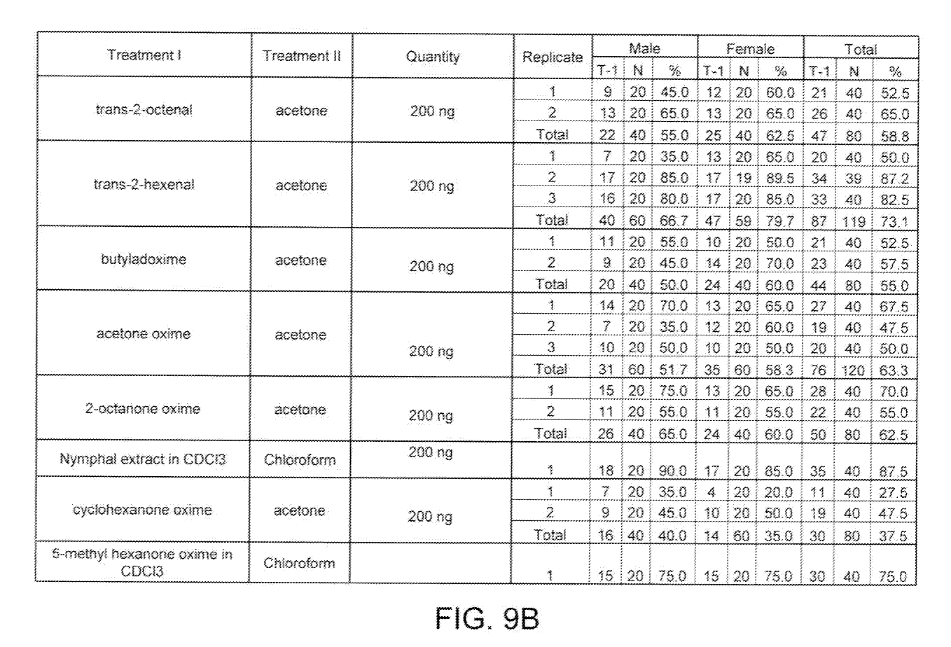

FIG. 9 represents an example of a two-choice test with bed bugs from the species Cimex lectularius. As set forth in FIG. 9, the “post NMR” sample exhibited 89.5% effectiveness when analyzed via a two-choice bioassay experiment.

### Example 5

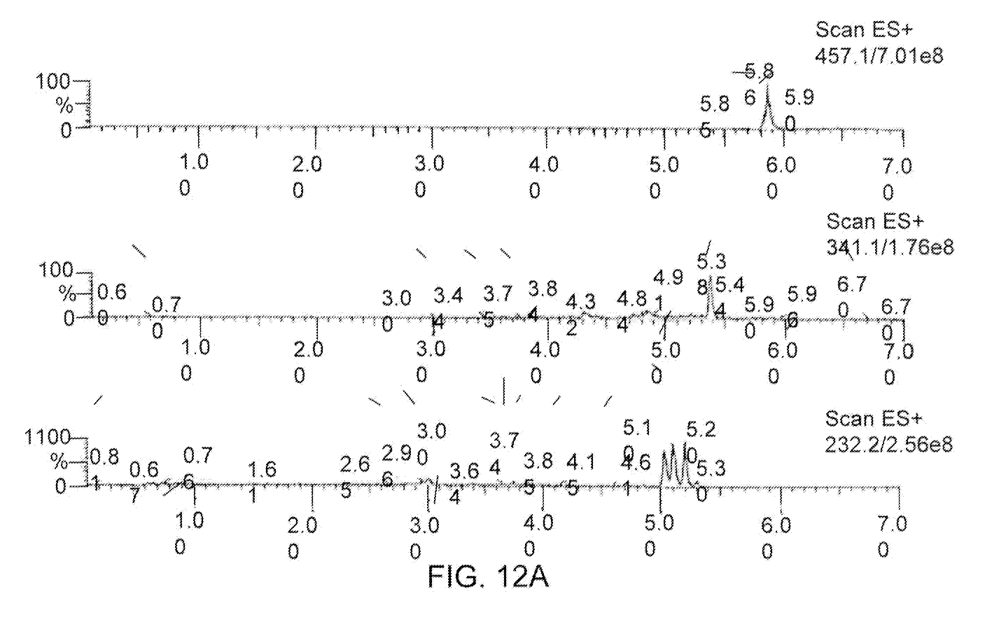

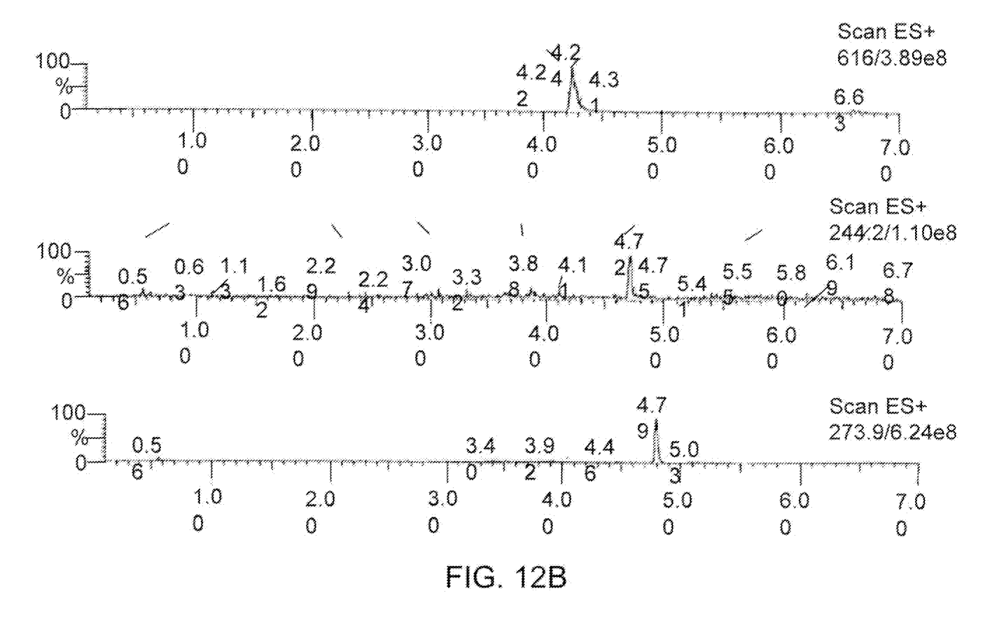

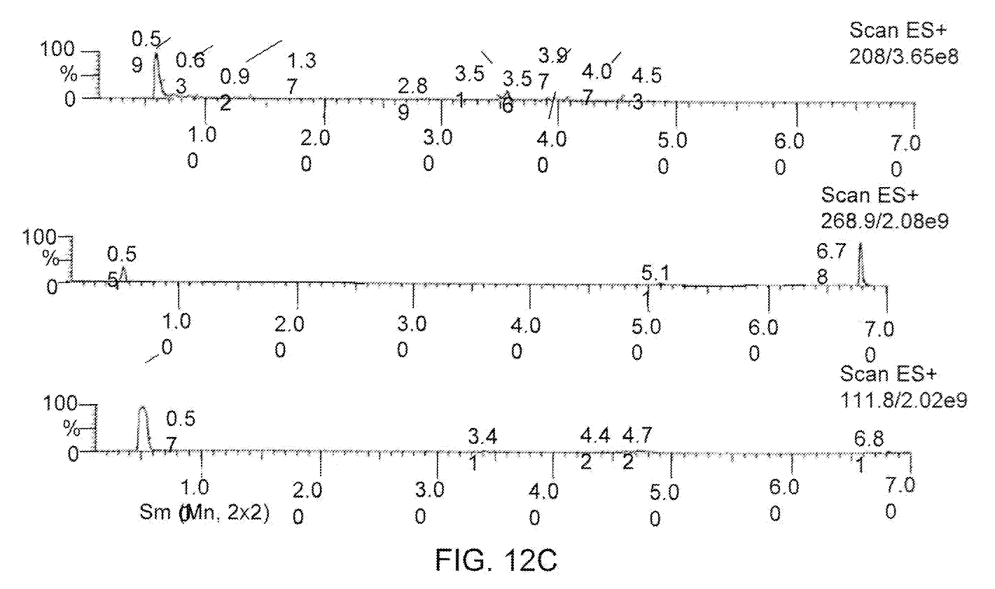

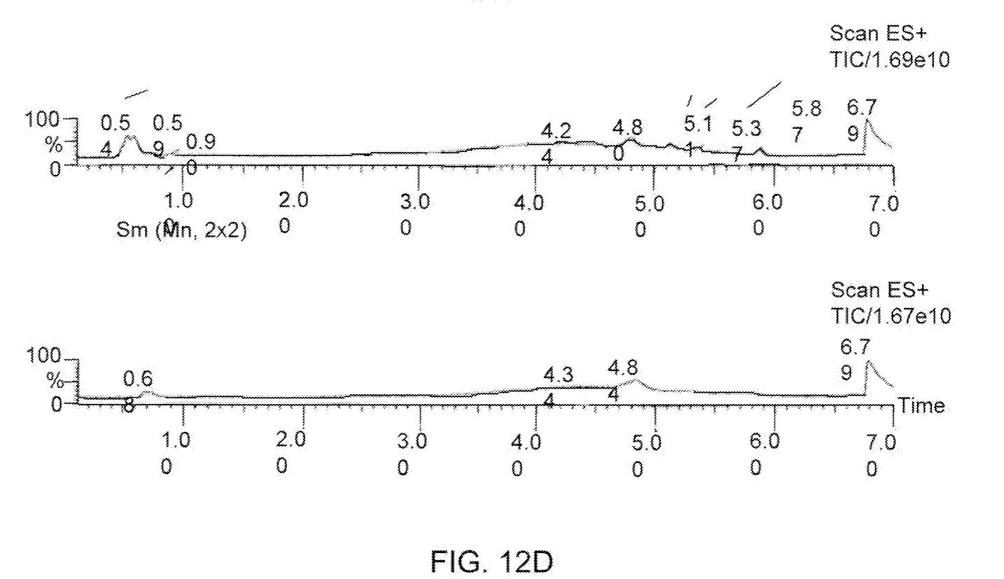

In this example, bed bug feces extract obtained from the methodology of Example 1 was evaluated by liquid chromatography-mass spectrometry (LC-MS). The full scan LC-MS scan is set forth in FIG. 11 (sample) and FIG. 12 (solvent blank). The LC-MS scans were run on the same feces extract sample as set forth in Example 4 (Carbon 13-NMR sample).

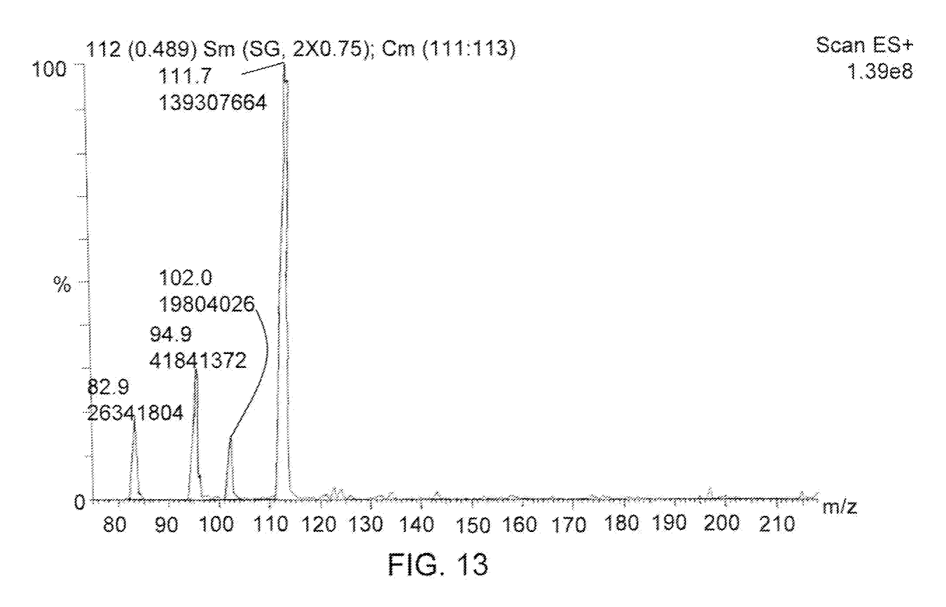

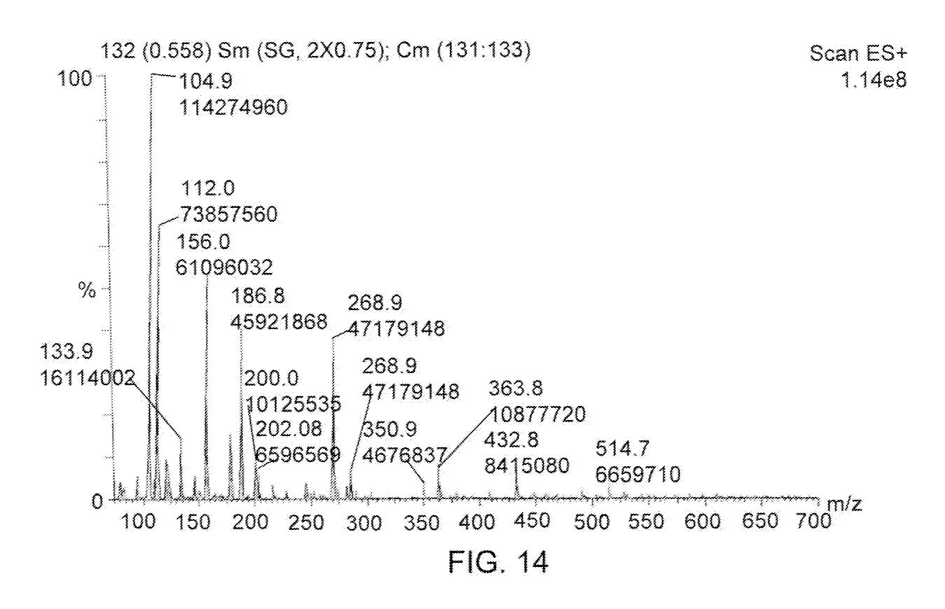

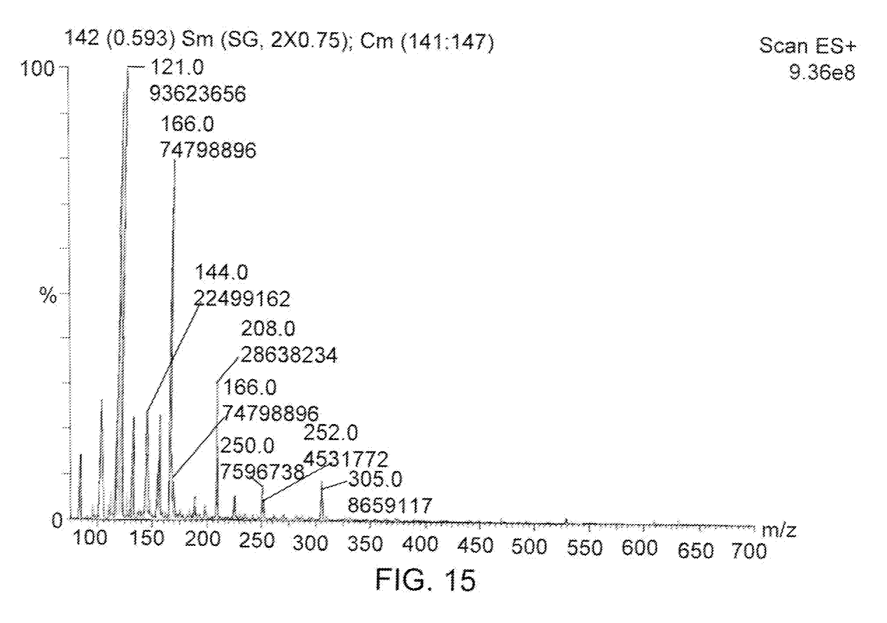

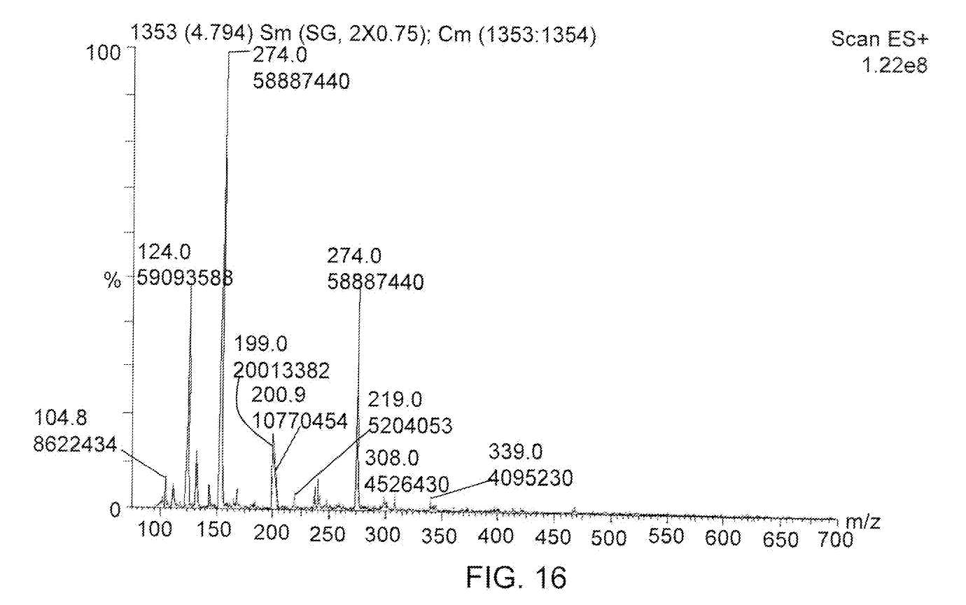

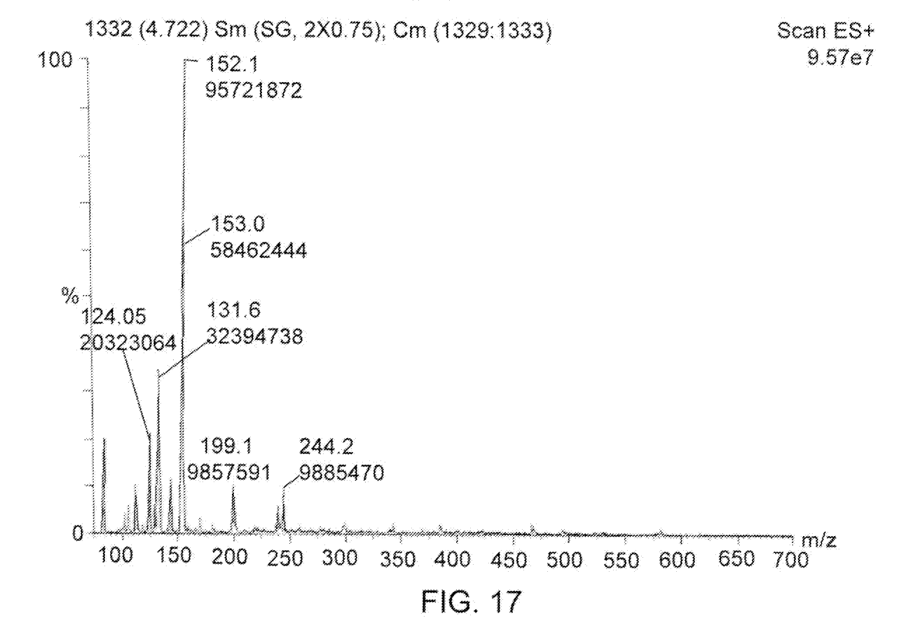

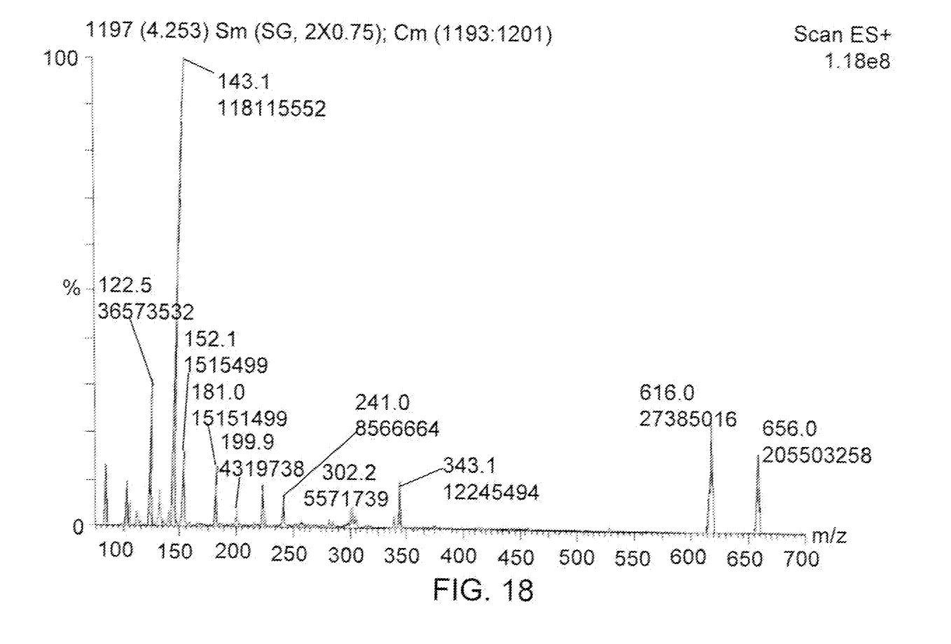

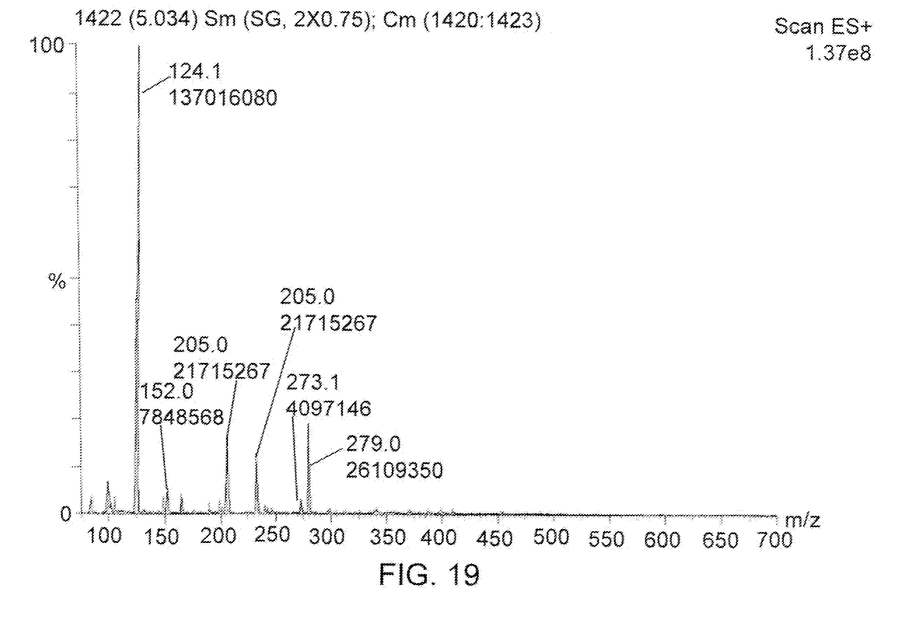

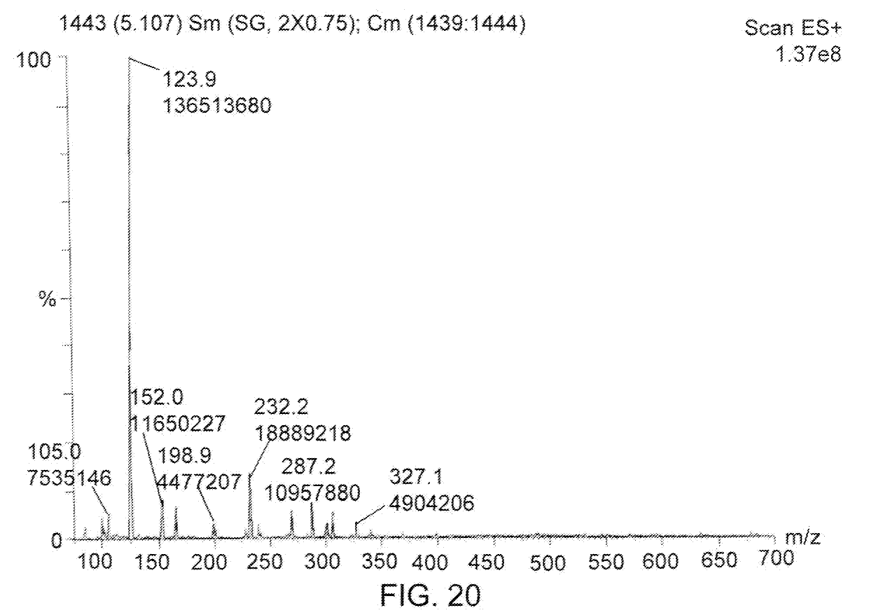

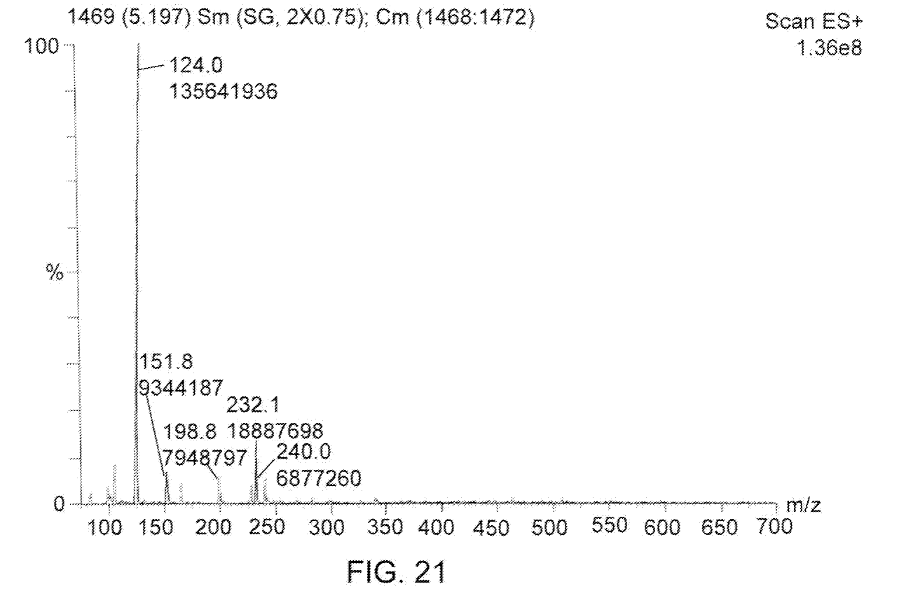

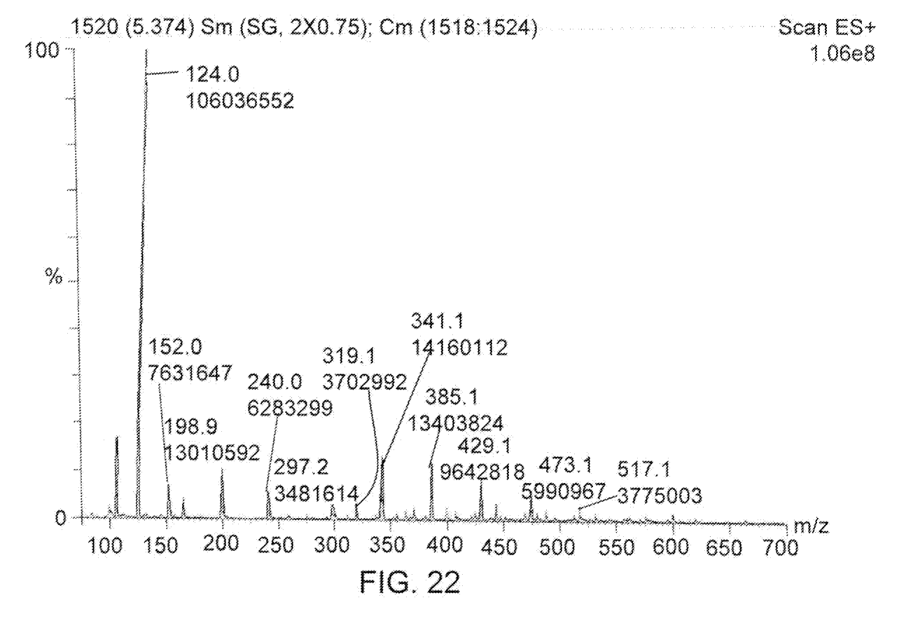

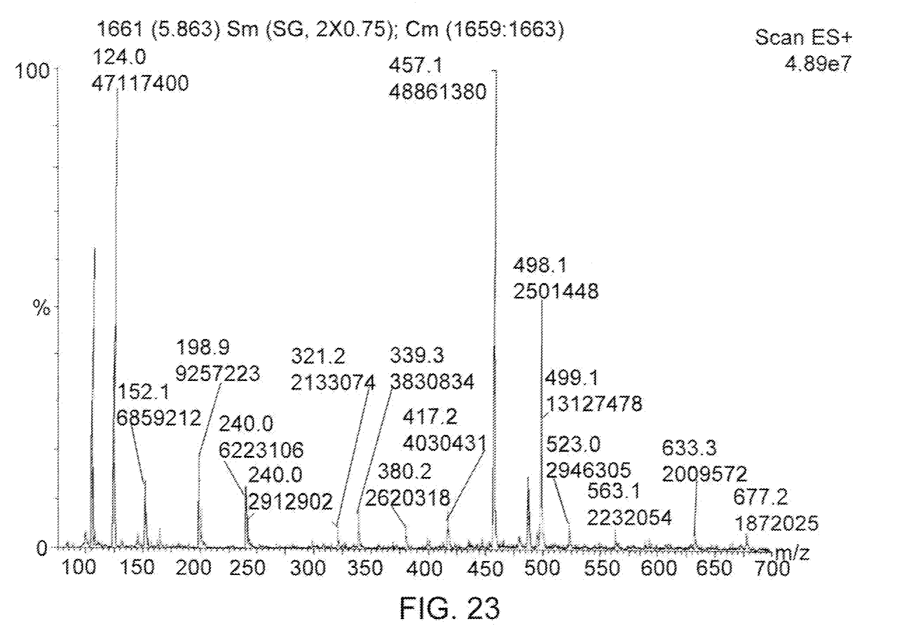

A bed bug feces extract was dissolved in methanol. The bed bug feces extract sample was analyzed prior to the removal of the solvent (methanol). The Liquid Chromatography was run in a solvent (mobile phase) of acetonitrile and water with addition of acetic acid. As set forth in FIGS. 11 (sample) and 12 (blank), the mass displayed is on the right hand side of the Figures and is underlined. Also provided are a full mass spectrum at a retention time at 0.489 min (FIG. 13), a full mass spectrum at a retention time at 0.558 min (FIG. 14), a full mass spectrum at a retention time at 0.593 min (FIG. 15), a full mass spectrum at a retention time at 4.794 min (FIG. 16), a full mass spectrum at a retention time at 4.722 min (FIG. 17), a full mass spectrum at a retention time at 4.253 min (FIG. 18), a full mass spectrum at a retention time at 5.034 min (FIG. 19), a full mass spectrum at a retention time at 5.107 min (FIG. 20), a full mass spectrum at a retention time at 5.197 min (FIG. 21), a full mass spectrum at a retention time at 5.374 min (FIG. 22), and a full mass spectrum at a retention time at 5.863 min (FIG. 23).

Having disclosed the instant subject matter, it should be apparent that many modifications, substitutions and variations of the present invention are possible in light thereof. It is to be understood that the present invention can be practiced other than as specifically described. Such modifications, substitutions and variations are intended to be within the scope of the present application. As used herein and in the following claims, articles such as “a”, “the” and so on can connote the singular or the plural of the object following.

1. A method for identifying an insect attractant, arrestant, and/or aggregation compound comprising identifying a compound in insect feces or fecal extract, wherein said compound is capable of attracting, arresting, and/or aggregating an insect.

2. The method of claim 1, wherein said insect attractant, arrestant, and/or aggregation compound is identified by an analytical technique selected from the group consisting of gas chromatography, nuclear magnetic resonance (NMR), mass spectroscopy, liquid chromatography-mass spectrometry (LC-MS), GC-MS, high performance liquid chromatography (HPLC), and combinations thereof.

3. The method of claim 1, wherein Carbon-13 NMR is used to identify said attractant, arrestant, and/or aggregation compound.

4. The method of claim 3, wherein said insect attractant, arrestant, and/or aggregation compound exhibits a peak at about δ 160 ppm when analyzed by Carbon-13 NMR.

5. The method of claim 3, wherein said insect attractant, arrestant, and/or aggregation compound exhibits a peak at about δ 159.453 ppm when analyzed by Carbon-13 NMR in acetone-d6 solvent.

6. The method of claim 5, wherein said insect attractant, arrestant, and/or aggregation compound identified by a peak at about δ 159.453 ppm when analyzed by Carbon-13 NMR in acetone-d6 solvent comprises a functional group selected from the group consisting of an oxime, hydrazone, imino, amidine, and imine functional group.

7. The method of claim 1, wherein prior to said identifying step, said insect feces is dissolved in a solvent thereby obtaining fecal extract.

8. The method of claim 7, wherein said solvent is an alcohol.

9. The method of claim 8, wherein said alcohol is methanol.

10. The method of claim 1, wherein said insect feces is from a large nymph insect.

11. A compound identified by the method of claim 1.

12. A compound identified by the method of claim 1, wherein said compound exhibits a peak at about δ 159.453 ppm when analyzed by Carbon-13 NMR in acetone-d6 solvent.

13. A composition comprising a compound identified by the method of claim 1.

14. The composition of claim 13 further comprising an insecticide.

15. The composition of claim 15, wherein said insecticide is selected from the group consisting of carbamates, pyrethroids, phenylpyrazoles, such as fipronil or ethiprole, chloronicotinyles, imidacloprid, acetamiprid, thiacloprid, thiamethoxam, nitenpyram, clothianidin, oxadiazines, anthranilic diamides, butenolides, sulfoximines, indoxacarb, and rynaxypyr.

16. The method of claim 1, wherein said insect is a bed bug.

17. A detection device for monitoring insect infestation, comprising
a first plate and a second plate, wherein the first plate and the second plate are separated by an internal spacing sized to permit entry into the detection device by one or more insects; and
a compound identified by the method of claim 1.

18. A composition capable of attracting an insect comprising a compound that exhibits a peak at about δ 159.453 ppm when analyzed by Carbon-13 NMR in acetone-d6 solvent and/or said compound exhibits a peak at about δ 160 ppm when analyzed by Carbon-13 NMR.

19. A composition of claim 18 wherein said insect is a bed bug.

20. A composition of claim 18 exhibiting a Carbon 13 NMR spectra as set forth in FIG. 3.
